(19) United States
(12) Patent Application Publication
(10) Pub. No.: US 2008/0019973 A1
(43) Pub. Date: Jan. 24, 2008

(54) Methods for Treating Ocular Inflammation by Neutralizing Cxcl10 Activity

(76) Inventors: Thomas E. Lane, Irvine, CA (US); Daniela J.J. Carr, Edmond, OK (US)

Correspondence Address: MCDERMOTT, WILL & EMERY, 4370 LA JOLLA VILLAGE DRIVE, SUITE 700, SAN DIEGO, CA, 92122, US

(21) Appl. No.: 10/549,482

(22) PCT Filed: Jul. 29, 2003
(86) PCT No.: PCT/US03/23838

§ 371 Date: Feb. 7, 2007

Related U.S. Application Data
(60) Provisional application No. 60/456,028, filed on Mar. 17, 2003.
(60) Provisional application No. 60/483,189, filed on Jun. 26, 2003.

(51) Int. Cl. A61K 39/395; A01K 67/00; C07K 16/24; C12Q 1/18; C12Q 1/68; G01N 33/53

(52) U.S. Cl. 424/145.1; 435/32; 435/6; 435/7.92; 800/9

(57) ABSTRACT

The invention provides a method of reducing ocular inflammation in an individual susceptible to ocular inflammation. The method involves administering to the individual an effective amount of a neutralizing agent specific for CXCL10. Also provided is a method for reducing spread of viral infection within ocular tissues of an individual susceptible to ocular viral infection, which involves administering to the individual an effective amount of a neutralizing agent specific for CXCL10. Further provided is a method of extending corneal graft survival following corneal transplantation in an individual, which involves administering to said individual an effective amount of a neutralizing agent specific for CXCL10. Additionally, the invention provides a method for identifying a compound for reducing ocular inflammation in an animal.

This invention was made with government support under grant number AI053108 awarded by the National Institutes of Health. The United States Government has certain rights in this invention.

### BACKGROUND OF THE INVENTION

When damage to a tissue of the body occurs, the body's immunologic response is usually inflammation. Inflammation can be caused, for example, by trauma, lack of blood supply, haemorrhage or infection. Generally, the process of inflammation includes the release of many components of the immune system, such as cytokines and chemokines, and attraction of immune system cells to the site of the damage. The overall effect of inflammation on the effected tissue is redness, swelling, heat, pain and loss of function.

Ocular inflammation, or inflammation of the eye, when unchecked can lead to permanent loss of vision. In fact, uveitis, or inflammation inside the eye, is the third leading cause of blindness in the United States, after diabetes and macular degeneration. Inflammation of the cornea (keratitis), which is the clear dome that covers the front part of the eye, also can lead to blindness. Keratitis occurs as a result of a wide variety of stimuli, but by far the most common is infection. The most common infectious cause of corneal blindness in the United States is herpes simplex virus.

Ocular herpes is a recurrent viral infection that affects an estimated 400,000 Americans with herpes. Studies have indicated that after an individual has had an initial outbreak of ocular herpes, he or she unfortunately has at least a 50 percent chance of recurrence. Those who lose vision to ocular herpes generally have experienced recurrent attacks. Ocular herpes typically causes inflammation on the surface of the cornea, referred to as Herpes Keratitis. In cases of more advanced infection, the deeper layers of the cornea can be affected. It is in these cases that Herpes Keratitis can lead to scars of the cornea, loss of vision, and even blindness. Less commonly, herpes can also infect and cause inflammation of the inside of the eye (Herpes Uveitis) or the retina (Herpes Retinitis).

The treatment of ocular inflammation in general depends on the location and the severity of the inflammation. With regard to ocular herpes, anti-viral medications generally have been used to successfully treat superficial corneal infection. However, these medications have been less effective for treating more advanced or widespread infection associated with severe inflammation. Steroid containing eye drops have been used as a treatment more severe inflammation, but unfortunately some individuals do not respond well or rapidly to treatment. These individuals can become afflicted by prolonged inflammation, which can cause permanent corneal scarring, ultimately lead to blindness.

Thus, there exists a need for treating ocular inflammation. The present invention satisfies this need and provides related advantages as well.

### SUMMARY OF THE INVENTION

The present invention provides a method of reducing ocular inflammation in an individual susceptible to ocular inflammation. The method involves administering to the individual an effective amount of a neutralizing agent specific for CXCL10. In one embodiment, the individual can be a mammal, such as a human. In another embodiment, the individual can be one that has an ocular infection, such as a microbial infection. In a further embodiment, the method can be used to reduce corneal inflammation.

The invention also provides a method for reducing spread of viral infection within ocular tissues of an individual susceptible to ocular viral infection. The method involves administering to the individual an effective amount of a neutralizing agent specific for CXCL10. In one embodiment, the individual can be a mammal, such as a human. In another embodiment, the individual can be one that has a herpes virus infection. In further embodiments, the method can be used to reduce spread of viral infection from the cornea to the retina, and from the cornea to the iris.

The invention further provides a method of extending corneal graft survival following corneal transplantation in an individual. The method involves administering to the individual an effective amount of a neutralizing agent specific for CXCL10. In one embodiment, a neutralizing agent is administered prior to corneal transplantation. In another embodiment, the neutralizing agent is administered after corneal transplantation. The neutralizing agent can be administered using a variety of routes, for example, interocularly or by release from an intraocular or periocular implant.

Also provided by the invention is a method for screening for a compound for reducing ocular inflammation in an animal. The method involves (a) providing a compound that is a neutralizing agent specific for CXCL10; and (b) determining the ability of the compound to reduce one or more indicia of ocular inflammation, wherein a compound that reduces one or more indicia of ocular inflammation is identified as a compound for reducing ocular inflammation in an animal. In one embodiment, the compound can be administered to an animal capable of exhibiting an index of ocular inflammation, such as a mammal. In another embodiment, the compound can be contacted with a synthetic or animal tissue capable of exhibiting an index of ocular inflammation. A variety of indicia of ocular inflammation can be used in the methods of the invention; exemplary indices include, but are not limited to, reduced corneal pathology, reduced leukocyte infiltration, reduced MIP-1α expression, reduced ICAM-1 expression, reduced CXCR3 expression, reduced RANTES expression, reduced viral antigen expression, reduced viral spread, increased survival and reduced neovascularization.

A neutralizing agent specific for CXCL10 used in any method of the invention can be, for example, a CXCL10 binding agent, such as an anti-CXCL10 antibody or fragment thereof.

### BRIEF DESCRIPTION OF THE DRAWINGS

FIG. 1 shows that neutralizing CXCL10 in HSV-1 infected mice prolongs survival.

FIG. 2 shows that neutralizing CXCL10 in HSV-corneal stroma (C), ciliary body (CB) and iris (I), with a representative anti-CXCL10 antibody treated mouse eye section shown in FIG. 2A and a representative control antibody treated mouse eye section shown in FIG. 2B, both at 40× magnification. Insets for FIGS. 2A and 2B depict ciliary body and stroma at 400× magnification.

FIG. 3 shows that neutralizing CXCL10 in HSV-1 infected mice reduces levels of MIP-1α and RANTES in the cornea/iris at day 5 (FIG. 3A) or day 7 (FIG. 3B) post-infection.

FIG. 4 shows that neutralizing CXCL10 in HSV-1 infected mice reduces HSV-1 antigen expression in the iris, with control antibody treated iris shown in FIG. 4A and anti-CXCL10 antibody treated iris shown in FIG. 4B.

FIG. 5 shows viral antigen expression in the tissue surrounding the optic nerve (ON) (FIG. 5A) or the choroid and photoreceptor layer of the retina (FIG. 5B) of mice six days post-infection by HSV-1.

FIG. 6 shows viral antigen expression in the ciliary body and nerve of mice six days post-infection by HSV-1 at 40× magnification (FIG. 6A) and 200× magnification (FIG. 6B).

FIG. 7 shows that neutralizing CXCL10 in HSV-1 infected mice results in reduced expression of MIP-1α and IFN-γ in the trigeminal ganglion at day 5 (FIG. 7A) and day 7 (FIG. 7B) post-infection.

FIG. 8 shows that neutralizing CXCL10 in HSV-1 infected mice results in reduced neovascularization.

### DETAILED DESCRIPTION OF THE INVENTION

This invention relates to the determination that neutralizing the activity of CXCL10 reduces ocular inflammation. Neutralizing CXCL10 provides an effective treatment option for ocular inflammation because it reduces both leukocyte infiltration into ocular tissues and expression of selective chemokines and ICAM-1 associated with inflammation-induced loss of tissue function following ocular insult. Thus, neutralizing CXCL10 targets a cause rather than a symptom of ocular inflammation. In addition, as disclosed herein, CXCL10 can be neutralized at the ocular surface, thus avoiding side effects caused by generalized immune suppression.

Chemokines are a large family of small, structurally related proteins that mediate a wide range of biological activities. In the immune system, chemokines have a critical role in mediating trafficking of leukocytes. Chemokines have other cellular roles, including regulation of growth, differentiation, and activation of leukocytes, as well as promoting effector functions of these cells, such as integrin activation, chemotaxis, superoxide radical production and granule enzyme release.

CXCL10 is a chemokine that directs migration of CXCR3-bearing cells, including NK cells and activated T cells (Loetscher et al., J. Exp. Med. 184:963 1996) and Qin et al., J. Clin. Invest. 101:746 (1998)). One role of NK cells and T cells in general is to facilitate the clearance of viruses, either by direct lysis of virally-infected cells or inhibition of viral replication through the release of soluble mediators such as IFN-γ (Engler et al., J. Gen. Virol. 55:25 (1981); Cantin et al., J. Virol. 69:4898 (1995) and Carr and Noisakran J. Virol. 76:9398 (2002)). However, many other immune system molecules influence the activity of NK cells and T cells in the immune response. For example, viral infection of the cornea, such as herpes virus infection, results in an explosive host response initiated by chemokine production including CXCL10, KC (murine CXCL1), macrophage inflammatory protein (MIP)-2, monocyte chemoattractant protein (MCP)-1, MIP-1α, and RANTES as well as pro-inflammatory cytokines including IL-6 (Su et al., J. Virol. 70:1277 (1996); Tumpey et al., J. Virol. 72:3705 (1998) and Fenton et al., Invest. Ophthalmol. Vis. Sci. 43:737 (2001)). A variety of cells within or proximal to the cornea are a likely source of these molecules. Such cells include resident Langerhans cells, keratocytes, and macrophages (Hendricks et al., J. Immunol. 148:2522 (1992); Mikloska et al., J. Infect. Dis. 177:827 (1997) and Brissette-Storkus et al., Invest. Ophthalmol. Vis. Sci. 43:2264 (2002)).

Neutrophils are attracted to MIP-2 and KC, resulting in infiltration into the cornea and subsequent secretion of inflammatory molecules, such as iNOS, TNF-α, IL-12, and IFN-γ (Diab et al., Infect. Immun. 67:2590 (1999); Tumpey et al., J. Virol. 70:898 (1996); Thomas et al., J. Immunol. 158:1383 (1997); Daheshia et al., Exp. Eye Res. 67:619 (1998) and Ellis and Beaman Nocardia asteroides. J. Leukoc. Biol. 72:373 (2002)). These soluble mediators elicit the expression or up-regulation of adhesion molecules including CD31 (platelet endothelial cell adhesion molecule 1, PECAM-1) and CD54 (ICAM-1) and the co-stimulatory molecule CD80 on resident Langerhans cells and keratocytes, resulting in the infiltration of CD4+ T cells (Tang and Hendricks, J. Exp. Med. 184:1435 (1996); Chen and Hendricks, J. Immunol. 160:5045 (1998); Seo et al., Eur. J. Immunol. 31:3318 (2001) and Niemialtowski and Rouse, J. Immunol. 148:1864 (1992)). It is the infiltration of these CD4+ T lymphocytes and the subsequent secretion of cytokines including IL-2 and IFN-γ along with other mediators independent of T cells (such as IL-12) that lead to the pathological manifestations of herpetic eye disease referred to as herpetic stromal keratitis (Russell et al., Invest. Ophthalmol. Vis. Sci. 25:938 (1984); Hendricks et al., J. Immunol. 149:3023 (1992); Bouley et al., J. Immunol. 155:3964 (1995); Tang, et al., J. Immunol. 158:1275 (1997) and Carr et al., DNA and Cell Biology. 21:467 (2002)). As described below, this complex cascade of events that follows immune system activation in the eye can surprisingly be blocked by neutralizing a single molecule, CXCL10. Importantly, such blocking can reduce collateral damage of the ensuing inflammatory response, saving the visual axis.

As disclosed herein, administration of an anti-CXCL10 antibody reduced the inflammatory response to ocular infection, as evidenced by a reduction in cellular infiltration as well as reduction in selective chemokine and ICAM-1 expression in the anterior segment of the eye (Example II); reduction in IFN-γ and MIP-1α expression in the trigeminal ganglion of the eye (Example VI); and reduction in neovascularization (Example VII). In addition, administration of the anti-CXCL10 antibody prolonged survival of HSV-1-infected mice (Example I).

Thus, in one embodiment, the invention provides a method for reducing ocular inflammation in an individual susceptible to ocular inflammation. The method involves administering to the individual an effective amount of a neutralizing agent specific for CXCL10.

As used herein, the term “ocular inflammation” means an inflammatory response occurring in a tissue of the eye. Such a response can include increased miosis, vasodilatation, compromise of the blood-aqueous barrier, increased opacity, protein infiltration into the aqueous humour, leukocyte infiltration and other well known physiological indicators of ocular inflammation. An inflammatory response also can include modulation of expression or release of a cytokine, chemokine, cell adhesion molecule or other immune system molecule associated with inflammation. Ocular inflammation can result from a variety of insults and conditions of the eye. Non-limiting examples of such insults and conditions include eye trauma or injury; exposure to excessive radiation, such as ultraviolet light; contact lens overuse; surgery; degeneration; in utero events such as infection and metabolic deficits; systemic drugs, such as adenine arabinoside and bisphosphonates; topical drug or preservative toxicity; chemical agents, such as alkalis, acids, organic solvents, pesticides, lacrimators, vesicants, ionic detergents, chemical warfare agents and the like; allergy; lack of blood supply; haemorrhage; infection, such as by a pathogenic organism, for example, a virus or bacterium; and various disorders. Examples of disorders that cause ocular inflammation include autoimmune disease, infectious disease, malignancies and graft rejection. Examples of infectious diseases that can cause ocular inflammation include microbial infection, such as viral infection, bacterial infection, fungal infection and parasitic infection. Examples of viruses that can cause ocular inflammation include members of the herpes family of viruses, such as herpes simplex virus, including HSV-1 and HSV-2, and varicella zoster virus (VZP); adenoviruses; entroviruses; and viruses that frequently infect immune-compromised individuals, such as cytomegalovirus (CMV), and the like. Examples of bacteria that can cause ocular inflammation include Staphylococcus spp., Streptococcus spp., Haemophilus influenzae, Pseudomonas aeruginosa, enteric Gram-negative bacilli, Moraxella lacunata, Acinetobacter spp., Neisseria gonorrhoeae, Branhamella catarrhalis, Clamydia trachomatis, and some anaerobic bacteria. Examples of fungi that can cause ocular inflammation include Aspergillus, Penicillium, Alternaria, Cladosporium, and Fusarium. Examples of parasites that can cause ocular inflammation include Acanthamoeba, such as A. culbertsoni, A. polyphaga, A. castellanii, A. healyi, (A. astronyxis), A. hatchetti, A. rhysodes, and Toxoplasma gondii. The term ocular inflammation is intended to encompass all types of ocular inflammation, such as serous, fibrinous, haemorrhagic, purulent or granulomatous inflammation, or a combination of these types. Any tissue of the eye can be susceptible to inflammation; non-limiting examples of tissues of the eye susceptible to inflammation include the vitreous, sclera, iris, pupil, lens, conjunctiva, vitreous, choroid, optic nerve, macula and retina. Therefore, examples of types of ocular inflammation that can be treated using a method of the invention include, but are not limited to, keratitis; conjunctivitis; episcleritis; scleritis; uveitis, whether anterior, intermediate, posterior, diffuse or more than one of these; maculopathies and retinal degeneration, such as Non-Exudative Age Related Macular Degeneration (ARMD), Exudative Age Related Macular Degeneration (ARMD), Choroidal Neovascularization, Diabetic Retinopathy, Central Serous Chorioretinopathy, Cystoid Macular Edema, Diabetic Macular Edema, Myopic Retinal Degeneration; inflammatory diseases, such as Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpiginous Choroiditis, Subretinal Fibrosis and Uveitis Syndrome, Vogt-Koyanagi-Harada Syndrome, Punctate Inner Choroidopathy, Acute Posterior Multifocal Placoid Pigment Epitheliopathy, Acute Retinal Pigement Epitheliitis, Acute Macular Neuroretinopathy; vascular and exudative diseases, such as Diabetic retinopathy, Central Retinal Arterial Occlusive Disease, Central Retinal Vein Occlusion, Disseminated Intravascular Coagulopathy, Branch Retinal Vein Occlusion, Hypertensive Fundus Changes, Ocular Ischemic Syndrome, Retinal Arterial Microaneurysms, Coat's Disease, Parafoveal Telangiectasis, Hemi-Retinal Vein Occlusion, Papillophlebitis, Central Retinal Artery Occlusion, Branch Retinal Artery Occlusion, Carotid Artery Disease (CAD), Frosted Branch Angiitis, Sickle Cell Retinopathy and other Hemoglobinopathies, Angioid Streaks, Familial Exudative Vitreoretinopathy; Eales Disease; traumatic, surgical and environmental disorders, such as Sympathetic Ophthalmia, Uveitic Retinal Disease, Retinal Detachment, Trauma, Retinal Laser, Photodynamic therapy, Photocoagulation, Hypoperfusion During Surgery, Radiation Retinopathy, Bone Marrow Transplant Retinopathy; proliferative disorders, such as Proliferative Vitreal Retinopathy and Epiretinal Membranes; infectious disorders, such as Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associate with HIV Infection, Uveitic Disease Associate with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, Myiasis; genetic disorders, such as Retinitis Pigmentosa, Systemic Disorders with Accosiated Retinal Dystrophies, Congenital Stationary Night Blindness, Cone Dystrophies, Stargardt's Disease And Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum; retinal injuries, such as Macular Hole, Giant Retinal Tear; retinal tumors, such as Retinal Disease Associated With Tumors, Congenital Hypertrophy Of The RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, and Intraocular Lymphoid Tumors.

A method of the invention can be used to reduce ocular inflammation associated with one or more particular tissues of the eye, or ocular inflammation associated with the eye in general. As a non-limiting example, a method of the invention can be applied to reducing corneal inflammation. A variety of corneal disorders or conditions can cause inflammation; such disorders or conditions include, but are not limited to, superficial punctate keratitis, corneal ulcer, herpes keratitis, herpes zoster ophthalmicus, Acanthomoeba keratitis, fungal keratitis, keratoconjuctivitis sicca, phlyctenular keratoconjunctivitis, interstitial keratitis, peripheral ulcerative keratitis, keratomalacia, keratoconus, bullous kertopathy and corneal transplantation.

As used herein, the term “reducing” when used in reference to ocular inflammation means arresting, decreasing or delaying onset of a physiological indicator or biochemical indicator of ocular inflammation. Physiological indicators include perceptible, outward or visible signs of ocular inflammation, increases in physical and chemical factors that correlate positively with ocular inflammation, as well as decreases in physical and chemical factors that correlate negatively with ocular inflammation. Exemplary physiological indicators of ocular inflammation include, but are not limited to, increased miosis, vasodilatation, compromise of the blood-aqueous barrier, increased opacity, protein infiltration into the aqueous humour, leukocyte infiltration. Biochemical indicators include those signs of ocular inflammation that are observable at the molecular level, such as the presence, absence or change in the amount of a chemokine, cytokine, adhesion molecule or other substance associated with ocular inflammation. Exemplary biochemical indicators of ocular inflammation include, but are not limited to, increased MIP-1α expression, increased CXCR3 expression, increased RANTES expression, increased IFNγ expression, increased ICAM-1 expression and increased neovascularization. One skilled in the art will be able to recognize specific physiological and biochemical indicators associated with ocular inflammation.

In one embodiment, a method of the invention involves administering a neutralizing agent specific for CXCL10 to an individual susceptible to ocular inflammation. As used herein, the term “individual susceptible to ocular inflammation,” means a human, veterinary animal or laboratory animal that exhibits, can be induced to exhibit, or is at high risk of exhibiting, ocular inflammation. Such veterinary or laboratory animals include, but are not limited to a non-human primate, horse, pig, feline, canine and rodent. An individual susceptible to ocular inflammation can exhibit, be induced to exhibit, or be at high risk of exhibiting ocular inflammation as a result of any of a variety of causes, including experimentally induced and non-experimentally induced causes.

With respect to veterinary animals, a variety of mammals are susceptible to and experience ocular inflammation. Such ocular inflammation can be severe and can lead to blindness if untreated. For example, fungal keratitis in horses is a well-known and frustrating clinical problem that accounts for at least 30% of equine infectious keratitis. Unfortunately, horses that have been treated with either topical antibiotics or corticosteroids are predisposed to fungal infection, because chronic topical antibiotic usage alters the microbial flora of the conjunctiva and corticosteroids suppress local immunity. Therefore, an exemplary veterinary animal that can be treated using a method of the invention is a horse.

An exemplary individual susceptible to ocular inflammation is one having or at risk of developing a bacterial or viral infection of an ocular tissue. Such an individual can be, for example, an individual infected with a herpes virus. Ocular herpes virus infection often leads to the formation of a painful sore on the eyelid or surface of the eye, which typically leads to corneal inflammation (keratitis). The infection can then spread deeper into the cornea, leading to a more serious condition called Stromal Keratitis, which causes the destruction of stromal cells via the immune system. With recurrence, ocular herpes virus infection can cause corneal scarring, which leads to loss of vision and potential blindness. Ocular herpes virus infection also can cause inflammation of the inner eye (uveitis) and/or retina (retinitis). Thus, an individual having a herpes virus infection is a candidate for treatment using a method of the invention for reducing ocular inflammation.

Methods for confirming that an individual has or is at risk of developing ocular inflammation, including those having ocular infections or having any of a variety of ocular conditions or systemic conditions associated with ocular inflammation, are well known to the skilled clinician. Thus, individuals for whom treatment using a method of the invention is appropriate can be readily identified. In one embodiment, an individual susceptible to ocular inflammation does not include an individual infected with HIV-1.

As disclosed in Example III, neutralizing CXCL10 in HSV-1 infected animals reduces spread of the virus from the cornea to the retina and iris. Therefore, the invention provides a method for reducing spread of viral infection within ocular tissues of an individual susceptible to ocular viral infection. The method involves administering to the individual an effective amount of a neutralizing agent specific for CXCL10. Reducing spread of a virus from one tissue to another can beneficially reduce ocular damage and loss of function resulting from viral infection and an ensuing inflammatory response in the virally infected tissue.

As used herein, the term “reducing spread of viral infection” means arresting, decreasing or delaying onset of a physiological indicator or biochemical indicator of spread of viral infection. Physiological indicators include perceptible, outward or visible signs of spread of viral infection and increases in physical and chemical factors that correlate positively with spread of viral infection, as well as decreases in physical and chemical factors that correlate negatively with spread of viral infection. Exemplary physiological indicators of spread of viral infection include, but are not limited to, presence of inflammation, presence of virus particles, presence of one or more viral antigens and the like. Biochemical indicators include those signs of spread of viral infection that are observable at the molecular level, such as the presence, absence or change in the amount of a chemokine, cytokine, adhesion molecule or other substance associated with viral infection in a tissue. One skilled in the art will be able to recognize specific physiological and biochemical indicators associated with viral infection and its spread.

The methods of the invention for reducing spread of viral infection involve administering a neutralizing agent specific for CXCL10 to an individual susceptible to ocular viral infection. As used herein, the term “individual susceptible to ocular viral infection” means a human, veterinary animal or laboratory animal has or is at high risk of having a viral infection of an ocular tissue. Such veterinary or laboratory animals include, but are not limited to a non-human primate, horse, pig, feline, canine and rodent. An individual susceptible to ocular viral infection can be one that is infected with, has been exposed to, or is likely to become infected with a virus that affects an ocular tissue. Examples of viruses that affect ocular tissues include, but are not limited to, herpes viruses, such as HSV-1, HSV-2, and varicella zoster virus (VZP); adenoviruses; entroviruses; and cytomegalovirus (CMV). In one embodiment, an individual susceptible to ocular viral infection is an individual that has a herpes virus infection. In another embodiment, an individual susceptible to ocular viral infection is an individual that has a viral infection of the cornea.

Spread of viral infection within ocular tissues of an individual can proceed from one or more ocular tissues initially infected to surrounding or adjacent non-infected tissues, whether directly through viral replication and trafficking or indirectly through another mechanism, such as via cells of the peripheral or central nervous system. For example, a viral infection of the cornea can spread to an uninfected portion of the cornea or to other tissues, such as the inner eye, retina and iris. Therefore, the methods of the invention for reducing spread of viral infection within ocular tissues offers a strategy for reducing the amount of ocular tissue subjected to viral infection and subsequent damage or loss of function.

Based on the discovery that neutralizing CXCL10 reduces corneal inflammation and neovascularization disclosed herein, the invention provides a method for extending corneal graft survival following corneal transplantation in an individual. The method involves administering to the individual an effective amount of a neutralizing agent specific for CXCL10. Neutralization of CXCL10 can promote corneal graft survival by reducing inflammation, neovascularization, and immune processes involving CXCL10 that contribute to graft rejection.

When used in reference to corneal transplantation, the term “individual” means the recipient of donor corneal tissue in a corneal transplantation procedure. The individual can be any of a variety of laboratory and veterinary animals, such as rodents, felines, horses, non-human primates and others, and can be a human individual. The individual receiving corneal transplantation can have ocular inflammation at the time of treatment, including ongoing ocular inflammation, or can have little or no detectable ocular inflammation. The individual further can be one considered to be at low risk or high risk for graft rejection. Individuals at high risk of graft rejection are generally those having ocular inflamation, for example, from injury or infection, and those with immune conditions of the eye surface, such as pemphigoid.

The methods of the invention are practiced to extend corneal graft survival following corneal transplantation. As used herein, the phrase “extending corneal graft survival” means that, on average, graft rejection is delayed or prevented. Thus, corneal graft survival is “extended” in a population when the number of months prior to allograft rejection is increased, on average, in the population, as compared to a corresponding population that was not treated with a neutralizing agent specific for CXCL10. Corneal graft survival also is extended in a population when the percentage of individuals with graft rejection decreases, on average, in the population, as compared to a corresponding population that was not treated with a neutralizing agent specific for CXCL10. The term graft rejection refers to the specific immunologic response of the host to the donor corneal tissue. A corneal graft that has suffered this immunologic response can survive or ultimately fail. Graft rejection therefore can refer to an immunological reaction that is “reversible” with medical therapy, or to an immunological reaction that is “irreversible.”

One skilled in the art uses established criteria to determine whether there is graft rejection. Rejection generally is evidenced as one or more pathologic events that involve the grafted cornea and progress toward the center of the graft but which do not effect the recipient cornea. Epithelial rejection is characterized by an epithelial rejection line appearing as a raised ridge of epithelium; subepithelial rejection is characterized by subepithelial infiltrates that resemble those seen in epidemic keratoconjunctivitis. Furthermore, stromal rejection is characterized by stromal infiltrates that progress toward the center of the graft, and endothelial rejection is characterized by at least one of the following: a Khodadoust line, keratic precipitates, stromal edema or aqueous cells. One skilled in the art understands that, in many cases, rejection is reversible with treatment such as topical dexamethasone; topical dexamethasone accompanied by subconjunctival dexamethasone injection and, if needed, accompanied by intravenous methylprednisone for several days. Rejection is considered irreversible when signs of rejection (rejection lines, subepithelial infiltrates, keratic precipitates, stromal infiltrates, stromal edema and aqueous cells) observed using slit-lamp examination fail to disappear; or there is abnormal graft thickness or loss of visual acuity.

As used herein, the term “neutralizing agent specific for CXCL10” means a substance that affects a decrease in the amount or rate of CXCL10 expression or activity. Such a substance can act directly, for example, by binding to CXCL10 and decreasing the amount or rate of CXCL10 expression or activity. A neutralizing agent specific for CXCL10 can also decrease the amount or rate of CXCL10 expression or activity, for example, by binding to CXCL10 in such a way as to reduce or prevent interaction of CXCL10 with a CXCL10 receptor; by binding to CXCL10 and modifying it, such as by removal or addition of a moiety; and by binding to CXCL10 and reducing its stability. A neutralizing agent specific for CXCL10 can also act indirectly, for example, by binding to a regulatory molecule or gene region so as to modulate regulatory protein or gene region function and affect a decrease in the amount or rate of CXCL10 expression or activity. Thus, a neutralizing agent specific for CXCL10 can act by any mechanism that results in decrease in the amount or rate of CXCL10 expression or activity.

A neutralizing agent specific for CXCL10 can be, for example, a naturally or non-naturally occurring macromolecule, such as a polypeptide, peptide, peptidomimetic, nucleic acid, carbohydrate or lipid. A neutralizing agent further can be an antibody, or antigen-binding fragment thereof, such as a monoclonal antibody, humanized antibody, chimeric antibody, minibody, bifunctional antibody, single chain antibody (scFv), variable region fragment (Fv or Fd), Fab or F(ab)2. A neutralizing agent can also be polyclonal antibodies specific for CXCL10. A neutralizing agent further can be a partially or completely synthetic derivative, analog or mimetic of a naturally occurring macromolecule, or a small organic or inorganic molecule.

A neutralizing agent specific for CXCL10 that is an antibody can be, for example, an antibody that binds to CXCL10 and inhibits binding to a CXCL10 receptor, or alters the activity of a molecule that regulates CXCL10 expression or activity, such that the amount or rate of CXCL10 expression or activity is decreased. As disclosed herein below, an antibody specific for CXCL10 was effective in prolonging survival of virally infected animals (Example I); in reducing ocular inflammation (Example II) and in reducing spread of virus in infected animals (Example III). An antibody useful in a method of the invention can be a naturally occurring antibody, including a monoclonal or polyclonal antibodies or fragment thereof, or a non-naturally occurring antibody, including but not limited to a single chain antibody, chimeric antibody, bifunctional antibody, complementarity determining region-grafted (CDR-grafted) antibody and humanized antibody or an antigen-binding fragment thereof.

A neutralizing agent specific for CXCL10 that is a nucleic acid can be, for example, an anti-sense nucleotide sequence, an RNA molecule, or an aptamer sequence. An anti-sense nucleotide sequence can bind to a nucleotide sequence within a cell and modulate the level of expression of CXCL10, CXCL0 receptor or modulate expression of another gene that controls the expression or activity of CXCL10.Similarly, an RNA molecule, such as a catalytic ribozyme, can bind to and alter the expression of the CXCL10 gene, or other gene that controls the expression or activity of CXCL10. An aptamer is a nucleic acid sequence that has a three dimensional structure capable of binding to a molecular target (Jayasena, Clinical Chemistry 45:9, 1628-1650 (1999)).

A neutralizing agent specific for CXCL10 that is a nucleic acid also can be a double-stranded RNA molecule for use in RNA interference methods. RNA interference (RNAi) is a process of sequence-specific gene silencing by post-transcriptional RNA degradation, which is initiated by double-stranded RNA (dsRNA) homologous in sequence to the silenced gene. A suitable double-stranded RNA (dsRNA) for RNAi contains sense and antisense strands of about 21 contiguous nucleotides corresponding to the gene to be targeted that form 19 RNA base pairs, leaving overhangs of two nucleotides at each 3′ end (Elbashir et al., Nature 411:494-498 (2001); Bass, Nature 411:428-429 (2001); Zamore, Nat. Struct. Biol. 8:746-750 (2001)). dsRNAs of about 25-30 nucleotides have also been used successfully for RNAi (Karabinos et al., Proc. Natl. Acad. Sci. USA 98:7863-7868 (2001). dsRNA can be synthesized in vitro and introduced into a cell by methods known in the art.

As used herein, the term “CXCL10 binding agent” means a neutralizing agent specific for CXCL10 that binds directly to CXCL10 to decrease CXCL10 amount or activity. The affinity of a CXCL10 binding agent for CXCL10 will generally be greater than about 10⁻⁵ M, such as greater than about 10⁻⁶ M, greater than about 10⁻⁸ M and greater than about 10⁻⁹ M. However, CXCL10 binding agents with low affinities or activities are also included within the meaning of the term where they can be made to specifically bind to CXCL10, for example, by modification. As used herein in reference to a CXCL10 neutralizing agent the term “specific” means that the agent alters the amount or rate of CXCL10 expression or activity without substantially altering expression or activity of other substances.

Various approaches can be used for identifying a neutralizing agent specific for CXLC10 useful in a method of the invention. One approach is to screen random candidate compounds for a neutralizing agent specific for CXLC10. Exemplary screening methods useful in this approach are described herein below. Another approach is to use the information available regarding the structure and function of CXCL10 to generate binding molecule populations from molecules known to function as chemokine binding molecules or known to exhibit or be capable of exhibiting binding affinity specific for CXCL10, such as fragments or mimetics of the CXCR3 receptor found on CD4+ T cells and NK cells. Yet another approach is to identify a naturally or non-naturally occurring antibody or fragment thereof specific for CXCL10.

Recombinant libraries of binding molecules can be used to identify a neutralizing agent specific for CXCL10 since large and diverse populations can be rapidly generated and screened with CXCL10. Recombinant libraries of expressed polypeptides useful for identifying a neutralizing agent specific for CXCL10 can be engineered using a variety of methods known in the art. Recombinant library methods similarly allow for the production of a large number of binding molecule populations from naturally occurring repertoires. Whether recombinant or otherwise, essentially any source of binding molecule population can be used so long as the source provides a sufficient size and diversity of different binding molecules to identify a neutralizing agent specific for CXCL10. If desired, a population of binding molecules useful for identifying a neutralizing agent specific for CXCL10 can be a selectively immobilized to a solid support as described by Watkins et al., Anal. Biochem. 256 (92): 169-177 (1998), which is incorporated herein by reference.

A phage expression library in which lysogenic phage cause the release of bacterially expressed binding molecule polypeptides is a specific example of a recombinant library that can be used to identify a neutralizing agent specific for CXCL10. In another type of phage expression library, large numbers of potential binding molecules can be expressed as fusion polypeptides on the periplasmic surface of bacterial cells. Libraries in yeast and higher eukaryotic cells exist as well and are similarly applicable to identifying a neutralizing agent specific for CXCL10 useful in a method of the invention. Those skilled in the art will know or can determine what type of library is useful for identifying a neutralizing agent specific for CXCL10.

A neutralizing agent specific for CXCL10 useful in a method of the invention can also be identified by using a purified CXCL10 polypeptide or peptide to produce antibodies. Such antibodies can be polyclonal or monoclonal, as well as antigen binding fragments thereof, including Fab, F(ab′)2, Fd and Fv fragments and the like.

Methods for preparing and isolating antibodies, including polyclonal and monoclonal antibodies, using peptide immunogens, are well known to those skilled in the art and are described, for example, in Harlow and Lane, Antibodies: A Laboratory Manual, Cold Spring Harbor Laboratory Press (1988), which is incorporated herein by reference. Producing monoclonal antibodies involves, in brief, fusion of spleen cells from a CXCL10 immunized mouse to an appropriate myeloma cell line to produce hybridoma cells. Cloned hybridoma cell lines can be screened using a labeled CXCL10 protein to identify clones that secrete anti-CXCL10. Hybridomas expressing anti-CXCL10 monoclonal antibodies having a desirable specificity and affinity can be isolated and utilized as a continuous source of the CXCL10 neutralizing agent.

Methods for raising polyclonal antibodies, for example, in a rabbit, goat, mouse or other mammal, are well known in the art (Harlow and Lane, supra, 1988). The production of anti-peptide antibodies commonly involves the use of host animals such as rabbits, mice, guinea pigs, or rats. If a large amount of serum is needed, larger animals such as sheep, goats, horses, pigs, or donkeys can be used. Animals are usually chosen based on the amount of antiserum required and suitable animals include rabbits, mice, rats, guinea pigs, and hamsters. These animals yield a maximum of 25 mL, 100-200 μL and 1-2 mL of serum per single bleed (Harlow and Lane, supra, 1988). Rabbits are useful for the production of polyclonal antisera, since they can be safely and repeatedly bled and produce high volumes of antiserum. Two injections two to four weeks apart with 15-50 μg of antigen in a suitable adjuvant such as, for example, Freund's Complete Adjuvant can be followed by blood collection and analysis of the antiserum.

Non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies are well known to those skilled in the art (Hoogenboom et al., U.S. Pat. No. 5,564,332, issued Oct. 15, 1996; Winter and Harris, Immunol. Today 14:243-246 (1993); Ward et al., Nature 341:544-546 (1989); Harlow and Lane, supra, 1988); Hilyard et al., Protein Engineering: A practical approach (IRL Press 1992); Borrabeck, Antibody Engineering, 2d ed. (Oxford University Press 1995); each of which is incorporated herein by reference).

Humanized antibodies can be constructed by conferring essentially any antigen binding specificity onto a human antibody framework. Humanized antibodies can reduce host immune responses when used therapeutically, in comparison to host immune response when a non-human antibody is used. Humanization of an antibody CXCL10 neutralizing agent can be accomplished, for example, by CDR-grafting as described in Fiorentini at al., Immunotechnology 3(1): 45-59 (1997), which is incorporated herein be reference. Briefly, CDR-grafting involves recombinantly splicing CDRs from a nonhuman CXCL10 neutralizing agent that is an antibody into a human framework region to confer binding activity onto the resultant grafted antibody, or variable region binding fragment thereof. Once the CDR-grafted antibody, or variable region binding fragment is made, binding affinity comparable to the nonhuman CXCL10 neutralizing agent can be reacquired by subsequent rounds of affinity maturation strategies known in the art. Humanization of antibodies that are CXCL10 neutralizing agents in the form of rabbit polyclonal antibodies can be accomplished by similar methods as described in Rader et al., J. Biol. Chem. 275(18): 13668-13676 (2000), which is incorporated herein be reference.

Humanization of a nonhuman CXCL10 neutralizing agent that is an antibody can also be achieved by simultaneous optimization of framework and CDR residues, which permits the rapid identification of co-operatively interacting framework and CDR residues, as described in Wu et al., J. Mol. Biol. 294(1): 151-162 (1999), which is incorporated herein by reference. Briefly, a combinatorial library that examines a number of potentially important framework positions is expressed concomitantly with focused CDR libraries consisting of variants containing random single amino acid mutations in the third CDR of the heavy and light chains. By this method, multiple Fab variants containing as few as one nonhuman framework residue and displaying up to approximately 500-fold higher affinity than the initial chimeric Fab can be identified. Screening of combinatorial framework-CDR libraries permits identification of monoclonal antibodies with structures optimized for function, including instances in which the antigen induces conformational changes in the monoclonal antibody. The enhanced humanized variants contain fewer nonhuman framework residues than antibodies humanized by sequential in vitro humanization and affinity maturation strategies known in the art.

Recombinant CXCL10 neutralizing agents that are antibodies include a wide variety of constructions ranging from simple expression and co-assembly of encoding heavy and light chain cDNAs to speciality constructs termed designer antibodies. Recombinant methodologies, combined with the extensive characterization of polypeptides within the immunoglobulin superfamily, and particularly antibodies, provides the ability to design and construct a vast number of different types, styles and specificities of binding molecules derived from immunoglobulin variable and constant region binding domains. Specific examples include chimeric antibodies, where the constant region of one antibody is substituted with that of another antibody, and humanized antibodies, described above, where the complementarity determining regions (CDR) from one antibody are substituted with those from another antibody.

Other recombinant versions of CXCL10 neutralizing agents that are antibodies include, for example, functional antibody variants where the variable region binding domain or functional fragments responsible for maintaining antigen binding is fused to an Fc receptor binding domain from the antibody constant region. Such variants are essentially truncated forms of antibodies that remove regions non-essential for antigen and Fc receptor binding. Truncated variants can be have single valency, for example, or alternatively be constructed with multiple valencies depending on the application and need of the user. Additionally, linkers or spacers can be inserted between the antigen and FC receptor binding domains to optimize binding activity as well as contain additional functional domains fused or attached to effect biological functions other than CXCL10 neutralization. Those skilled in the art will know how to construct recombinant CXCL10 neutralizing agents that are antibodies specific for CXCL10 in light of the art knowledge regarding antibody engineering and given the guidance and teachings herein. A description of recombinant antibodies, functional fragments and variants and antibody-like molecules can be found, for example, in Antibody Engineering, 2nd Edition. (Carl A. K. Borrebaeck, Ed.) Oxford University Press, New York, (1995).

Additional functional variants of antibodies that can be used as CXCL10 neutralizing agents that are antibodies include antibody-like molecules other than antigen binding-Fc receptor binding domain fusions. For example, antibodies, functional fragments and fusions thereof containing a Fc receptor binding domain can be produced to be bispecific in that one variable region binding domain exhibits binding activity for one antigen and the other variable region binding domain exhibits binding activity for a second antigen. Such bispecific CXCL10 neutralizing agents can be advantageous in the methods of the invention because a single bispecific antibody will contain two different target antigen binding species. Therefore, a single molecular entity can be administered to achieve neutralization of CXCL10.

An CXCL10 neutralizing agent that is an antibody can also be an immunoadhesion or bispecific immunoadhesion. Immunoadhesions are antibody-like molecules that combine the binding domain of a non-antibody polypeptide with the effector functions of an antibody of an antibody constant domain. The binding domain of the non-antibody polypeptide can be, for example, a ligand or a cell surface receptor having ligand binding activity. Immunoadhesions for use as CXCL10 neutralizing agents can contain at least the Fc receptor binding effector functions of the antibody constant domain. Specific examples of ligands and cell surface receptors that can be used for the antigen binding domain of an immunoadhesion neutralizing agent include, for example, a T cell or NK cell receptor such as the CXCR3 receptor that recognizes CXCL10. Other ligands and ligand receptors known in the art can similarly be used for the antigen binding domain of an immunoadhesion neutralizing agent specific for CXCL10. In addition, multivalent and multispecific immunoadhesions can be constructed for use as CXCL10 neutralizing agents. The construction of bispecific antibodies, immunoadhesions, bispecific immunoadhesions and other heteromultimeric polypeptides which can be used as CXCL10-specific neutralizing agents is the subject matter of, for example, U.S. Pat. Nos. 5,807,706 and 5,428,130, which are incorporated herein by reference.

A CXCL10 neutralizing agent that is an antibody specific for CXCL10 be raised using as an immunogen a substantially purified CXCL10 protein, which can be prepared from natural sources or produced-recombinantly, or a peptide portion of a CXCL10 protein including synthetic peptides. A non-immunogenic peptide portion of a CXCL10 protein can be made immunogenic by coupling the hapten to a carrier molecule such bovine serum albumin (BSA) or keyhole limpet hemocyanin (KLH), or by expressing the peptide portion as a fusion protein. Various other carrier molecules and methods for coupling a hapten to a carrier molecule are well known in the art (see Harlow and Lane, supra, 1988; see, also, Hermanson, Bioconjugate Techniques, Academic Press, 1996, which is incorporated herein by reference). As described above, an CXCL10 neutralizing agent can also be an antibody raised against a regulatory molecule that modulates CXCL10 expression or activity rather than against CXCL10 directly.

A neutralizing agent specific for CXLC10, such as a CXCL10 binding agent, can be a compound that reduces or blocks binding of CXCL10 to its receptor, thus selectively inhibiting or decreasing normal signal transduction through the receptor. Therefore, a neutralizing agent specific for CXCL10 can be a CXCL10 receptor antagonist. Such a receptor antagonist can be a competitive, noncompetitive, or uncompetitive antagonist, and further can function in a reversible or irreversible manner. A CXCL10 receptor antagonist can act by any antagonistic mechanism, such as by binding a CXCL10 receptor or CXCL10, thereby inhibiting binding between CXCL10 and CXCL10 receptor. A CXCL10 receptor antagonist can also act, for example, by inhibiting the binding activity of CXCL10 or signaling activity of CXCL10 receptor. For example, a CXCL10 receptor antagonist can act by altering the state of phosphorylation or glycosylation of CXCL10 receptor.

The receptor for CXCL10 has been identified as CXCR3, which is a G-protein coupled receptor. As is well known in the art, activation of a G-protein coupled receptor leads to G-protein coupled signal transduction, which can be measured as a readout for receptor activation. Various assays, including high throughput. automated screening assays, to identify alterations in G-protein coupled signal transduction pathways are well known in the art. Various screening assays that measure Ca⁺⁺, cAMP, voltage changes and gene expression are reviewed, for example, in Gonzalez et al., Curr. Opin. in Biotech. 9:624-631 (1998); Jayawickreme et al., Curr. Opin. Biotech. 8:629-634 (1997); and Coward et al., Anal. Biochem. 270:2424-248 (1999). Yeast cell-based bioassays for high-throughput screening of drug targets for G-protein coupled receptors are described, for example, in Pausch, Trends in Biotech. 15:487-494 (1997). A variety of cell-based expression systems, including bacterial, yeast, baculovirus/insect systems and mammalian cells, useful for detecting G-protein coupled receptor agonists and antagonists are reviewed, for example, in Tate et al., Trends in Biotech. 14:426-430 (1996).

A variety of well-known assays can be used to determine if a compound is a CXCL10 receptor antagonist. For example, in a signaling assay, a CXCL10 receptor is contacted with one or more candidate compounds under conditions wherein the CXCL10 receptor produces a predetermined signal in response to an agonist, such as CXCL10 or CXCL9. In response to CXCL10 receptor activation, a predetermined signal can increase or a decrease from an unstimulated CXCL10 receptor baseline signal. A predetermined signal is an increasing signal, for example, when the amount of detected second messenger molecule is increased in response to CXCL10 receptor activation. A predetermined signal is a decreasing signal, for example, when the detected second messenger molecule is destroyed, for example, by hydrolysis, in response to CXCL10 receptor activation. A predetermined signal in response CXCL10 receptor activation can therefore be an increase in a predetermined signal that correlates with increased CXCL10 receptor activity, or a decrease in a predetermined signal that correlates with increased CXCL10 receptor activity. Thus, a signaling assay can be performed to determine whether a candidate compound is a CXCL10 receptor antagonist. In such a signaling assay, a CXCL10 receptor is contacted with one or more candidate compounds under conditions wherein the CXCL10 receptor produces a predetermined signal in response to an agonist, such as CXCL10, and a compound is identified that reduces production of the predetermined signal.

Assays to detect and measure G-protein-coupled signal transduction can involve first contacting a sample containing CXCL10 receptor, such as an isolated cell, membrane or artificial membrane, such as a liposome or micelle, with a detectable indicator. A detectable indicator can be any molecule that exhibits a detectable difference in a physical or chemical property in the presence of the substance being measured, such as a color change. Calcium indicators, pH indicators, and metal ion indicators, and assays for using these indicators to detect and measure selected signal transduction pathways are described, for example, in Haugland, Molecular Probes Handbook of Fluorescent Probes and Research Chemicals, Sets 20-23 and 25 (1992-94).

Another type of signaling assay involves determining changes in gene expression in response to a CXCL10 receptor antagonist. A variety of signal transduction pathways contribute to the regulation of transcription in animal cells by stimulating the interaction of transcription factors with genetic sequences termed response elements in the promoter regions of responsive genes. Assays for determining the interaction of transcription factors with promoter regions to stimulate gene expression are well known to those skilled in the art and are commercially available. Exemplary gene expression assays are those that involve transducing cells with a promoter-reporter nucleic acid construct such that a readily detectable protein such as β-lactamase, luciferase, green fluorescent protein or β-galactosidase-will be expressed in response to contacting CXCL10 with an agonist, such as CXCL10. Compounds identified in such gene expression assays can act either at the level of the cell surface, by modulating the activity of a CXCL10 receptor, the activity of a component of the CXCL10 receptor signal cascade or the activity of factors that modulate transcription of a CXCL10 controlled gene.

A binding assay can be performed to identify compounds that are CXCL10 receptor antagonists. In such an assay, a CXCL10 receptor can be contacted one or more candidate compounds under conditions in which CXCL10 binds to the CXCL10 receptor and a compound that reduces binding of CXCL10 to CXCL10 receptor can be identified. Contemplated binding assays can involve detectably labeling a candidate compound, or competing an unlabeled candidate compound with a detectably labeled CXCL10. A detectable label can be, for example, a radioisotope, fluorochrome, ferromagnetic substance, or luminescent substance. Exemplary radiolabels useful for labeling compounds include ¹²⁵I, ¹⁴C and ³H. Methods of detectably labeling organic molecules, either by incorporating labeled amino acids into the compound during synthesis, or by derivatizing the compound after synthesis, are known in the art.

In order to determine whether a candidate compound decreases binding of detectably labeled CXCL10 to CXCL10 receptor, the amount of binding of a given amount of the detectably labeled CXCL10 is determined in the absence of the candidate compound. Generally the amount of detectably labeled CXCL10 will be less than its K_{d}, for example, 1/10 of its K_{d}. Under the same conditions, the amount of binding of the detectably labeled CXCL10 in the presence of the candidate compound is determined. A decrease in binding due to a candidate compound is evidenced by at least 2-fold less, such as at least 10-fold to at least 100-fold less, such as at least 1000-fold less, binding of detectably labeled CXCL10 to CXCL10 receptor in the presence of the candidate compound than in the absence of the candidate compound.

A variety of other low- and high-throughput assays suitable for detecting selective binding interactions between a receptor and a ligand are known in the art. Such assays include, for example, fluorescence correlation spectroscopy (FCS) and scintillation proximity assays (SPA) reviewed in Major, J. Receptor and Signal Transduction Res. 15:595-607 (1995); and in Sterrer et al., J. Receptor and Signal Transduction Res. 17:511-520 (1997)). Binding assays can be performed in any suitable assay format including, for example, cell preparations such as whole cells or membranes that contain CXCL10 receptor, or substantially purified CXCL10 receptor polypeptide, either in solution or bound to a solid support. Signaling and binding assays, including those described above for identifying a neutralizing agent specific for CXCL10 that is a CXCL10 receptor antagonist, typically involve detection of a predetermined signal. A predetermined signal is a readout, detectable by any analytical means, that is a qualitative or quantitative indication of activation of G-protein-dependent signal transduction through CXCL10 receptor.

A variety of cell types, including naturally occurring and genetically engineered cells, can be used in an in vitro assay to detect CXCL10 receptor activity or a downstream effect of CXCL10 receptor activity. Naturally occurring and in vitro cultured cells that express endogenous CXCL10 receptor include, for example, effector/memory T cells, minor subsets of B and NK cells (Qin et al. J Clin Invest. 101(4):746-54 (1998)), plasmacytoid dendritic cells (Cella et al. Nat. Med. 5:919-923 (1999)), eosinophils (Jinquan et al. J. Immunol. 165:1548-1556 (2000), GM-CSF activated CD34 (+) hematopoietic progenitors (Jinquan et al. Blood 96:1230-1238 (2000)). CXCL10 is expressed in high levels in a variety of types of long-term cultured T cells, including subsets marked by expression of CD4, CD8, α/β-TCR or γ/δ-TCR. Other naturally occurring cells and cell lines that express CXCR3 can be identified by those skilled in the art using methods disclosed herein and other methods well known in the art.

Cells expressing CXCR3 can be prepared using a variety of methods. Recombinant expression can be advantageous in providing a higher level of expression of CXCR3 than is found endogenously, and also allows expression in cells or extracts in which expression is not normally found. A recombinant nucleic acid expression construct generally contains a constitutive or inducible promoter of RNA transcription appropriate for the host cell or transcription-translation system, operatively linked to a nucleotide sequence that encodes a polypeptide corresponding to CXCR3 or an active fragment thereof. The expression construct can be DNA or RNA, and optionally can be contained in a vector, such as a plasmid or viral vector.

The nucleotide and amino acid sequences of human CXCR3 are available to one skilled in the art, for example, in the NCBI database as GenBank Accession No.

NM_{—}001504.1. Other human CXCR3 nucleotide and polypeptide sequences are available from GenBank, as are othologous CXCR3 sequences from rat, mouse and other species. Any of these CXCR3 nucleotide sequences can be used to recombinantly express a CXCR3 in an assay for confirming the activity of a neutralizing agent specific for CXCL10. The nucleotide and amino acid sequences of human CXCL10 are available to one skilled in the art, for example, in the NCBI database as GenBank Accession No. X02530. One skilled in the art can recombinantly express desired levels of CXCL10 or its receptor using routine laboratory methods, described, for example, in standard molecular biology technical manuals, such as Sambrook et al., supra (1992) and Ausubel et al., supra (1998).

Exemplary host cells that can be used to express recombinant CXCL10 or CXCR3 include isolated mammalian primary cells; established mammalian cell lines, such as COS, CHO, HeLa, NIH3T3, HEK 293-T and PC12; amphibian cells, such as Xenopus embryos and oocytes; and other vertebrate cells. Exemplary host cells also include insect cells such as Drosophila, yeast cells such as S. cerevisiae, S. pombe, and Pichia pastoris and prokaryotic cells such as E. coli.

The number of different candidate compounds to screen in a particular assay can be determined by those skilled in the art, and can be 2 or more, such as 5, 10, 15, 20, 50 or 100 or more different compounds. For certain applications, such as when a library of random compounds is to be screened, and for automated procedures, it may be desirable to screen 10³ or more compounds, such as 10⁵ or more compounds, including 10⁷ or more compounds.

Compounds for screening can be contained within large libraries of compounds, such as when high-throughput in vitro screening formats are used. Methods for producing large libraries of chemical compounds, including simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources.

Compounds can be screened individually or in pools of a few, tens or hundreds of compounds. Therefore, a library of compounds can be screened sequentially, in a multi-sample format, in which each sample receives one compound, or multiplexed format, in which each sample receives more than one compound. A neutralizing agent specific for CXCL10 can also be identified from a large population of candidate compounds by methods well known in the art.

A neutralizing agent specific for CXCL10 can be labeled so as to be detectable using methods well known in the art (Hermanson, supra, 1996; Harlow and Lane, supra, 1988; chapter 9). For example, a neutralizing agent specific for CXCL10 can be linked to a radioisotope or therapeutic agent by methods well known in-the art. A neutralizing agent that directly binds CXCL10 linked to a radioisotope or other moiety capable of visualization can be useful to diagnose or stage the progression of a clinical stage of secondary tissue degeneration associated with CNS injury that is characterized by the organ or tissue-specific presence or absence of CXCL10.

As disclosed herein, neutralizing CXCL10 in HSV-1 infected animals results in a reduction in ocular inflamation as evidenced by both reduced immune cell infiltration, reduced chemokine and ICAM-1 expression and improved pathology (Example II), as well as reduced neovascularization (Example VII). Based on these results, the invention provides a method for screening for a compound for reducing ocular inflammation in an animal. The method involves (a) providing a compound that is a neutralizing agent specific for CXCL10; and (b) determining the ability of said compound to reduce one or more indicia of ocular inflammation, wherein a compound that reduces one or more indicia of ocular inflammation is identified as a compound for reducing ocular inflammation in an animal.

As also disclosed herein, neutralizing CXCL10 can prolong survival of virally infected mammals (Example I), as well as reduce viral spread (Example III). In addition, as disclosed herein, a neutralizing agent specific for CXCL10 can be effective in extending corneal graft survival. Thus a compound for reducing ocular inflammation also can be used in a method for prolonging survival; in a method of reducing viral spread; and in a method of extending corneal graft survival following corneal transplantation. Alternatively, a compound for prolonging survival, such as survival of an animal susceptible to inflammation or survival of an ocular tissue susceptible to inflammation, either in vivo, ex vivo or in vitro, can be identified by (a) providing a compound that is a neutralizing agent specific for CXCL10; and (b) determining the ability of said compound to prolong survival, wherein a compound that increases one or more indicia of survival is identified as a compound for prolonging survival.

Similarly, a compound for reducing spread of viral infection with ocular tissues of an individual, can be identified by (a) providing a compound that is a neutralizing agent specific for CXCL10; and (b) determining the ability of said compound to reduce one or more indicia of viral spread, wherein a compound that reduces one or more indicia of viral spread is identified as a compound for reducing spread of viral infection.

Moreover, a compound for extending corneal graft survival following corneal transplantation in an individual can be identified by (a) providing a compound that is a neutralizing agent specific for CXCL10; and (b) determining the ability of said compound to reduce one or more indicia of graft rejection, wherein a compound that reduces one or more indicia of graft rejection is identified as a compound for extending corneal graft survival following corneal transplantation in an individual. Exemplary indicia of graft rejection are described herein.

In the screening methods of the invention, a compound provided is a neutralizing agent specific for CXCL10. The neutralizing agent can be one that is known, or one identified using any of a variety of methods, including the methods described herein for designing and screening to identify or confirm the activity of a neutralizing agent specific for CXCL10.

A compound provided in a method of the invention can be provided to a an in vitro, ex vivo or in vivo system predictive of an inflammatory response of the eye. In vitro models of the eye that can exhibit one or more indicia of ocular inflammation are generally synthetic, or man-produced, tissues that exhibit morphological and growth characteristics similar to in vivo or ex vivo tissue, and can produce physiological and/or biochemical indicators of inflammation. A commercially available example of an in vitro model suitable for confirming the ability of a CXCL10 neutralizing agent to reduce one or more indicia of ocular inflammation is the EPIOCULAR™ Model (MatTeck Corporation, Ashland, Mass.), a synthetic tissue with a cornea-like three dimensional structure.

EX vivo models of the-eye that can exhibit one or more indicia of ocular inflammation are generally tissues removed from the eye of an animal and maintained under conditions in which physiological and/or biochemical indicators of inflammation can be exhibited. The Bovine Corneal Opacity and Permeation assay (BCOP) is a well-known example of an ex vivo model for ocular inflammation. The assay is performed by removing the corneas from cow eyes (available as a by-product of a slaughter house) and placing the cornea between two chambers. The test chemical solution is then applied to the upper chamber. Subsequently, the cornea is can be removed and tested for an indicia of inflammation, such as opacity, influx of immune system cells, presence of biochemical indicators of inflammation, and the like.

There are a variety of well known in vivo models that exhibit one or more indicia of ocular inflammation. As disclosed herein, in Examples I through VI, a rodent is a suitable in vivo model for confirming the ability of a CXCL10 neutralizing agent to reduce ocular inflammation. Other exemplary animal models of ocular inflammation include, but are not limited to, those described in Suzuki et al. Cornea 21(8):812-7 (2002); Smith et al. Curr. Eye. Res. 21(5):906-12 (2000); Hume et al. Curr. Eye. Res. 19(6):525-32 (1999); and Cole et al. Curr. Eye. Res. 17(7):730-735 (1998). Such models can be adapted for use in a variety of research animals, as desired.

The screening methods of the invention involve determining the ability of a neutralizing agent specific for CXCL10 to reduce one or more indicia of ocular inflammation. Examples of indicia of ocular inflammation that can be determined using well-known methods include, but are not limited to, reduced corneal pathology; reduced leukocyte infiltration; reduced ulceration of the cornea; reduced development of opacity of the cornea; reduced swelling; reduced miosis; reduced vasodilatation; reduced compromise of the blood-aqueous barrier; reduced protein infiltration into the aqueous humour; reduced development of intraocular pressure; reduced expression or release of cytokines and/or chemokines having up-regulation or high levels of expression associated with ocular inflammation, such as reduced MIP-1α expression, reduced CXCR3 expression, reduced RANTES expression, reduced IFNγ expression; reduced expression of immune system molecules associated with ocular inflammation, such as reduced expression of ICAM-1; reduced viral antigen amount; reduced viral spread, such as reduced spread from cornea to retina and/or iris; increased survival; and reduced neovascularization.

Animal models of graft rejection are well known to those skilled in the art. Such models can parallel the physiological and biochemical indicators of graft rejection in humans, and thus can be useful for predicting the effect of a neutralizing agent specific for CXCL10 on extending corneal graft survival following corneal transplantation. The models typically involve implanting a graft on the eye of the animal under anesthesia and observing indicia of graft rejection, such as the presence of a rejection line. A rejection line is a region of destruction of donor epithelium in which the resulting epithelial damage is covered by host epithelium that grows inward from the remaining host cornea and limbus to cover the graft.

Indicia of graft rejection can be observed in one or more corneal layers, such as the epithelium, stroma, and endothelium. Epithelial rejection can be indicated by an irregular, elevated epithelial rejection line that stains with fluorescein or rose bengal. The rejection line typically progresses rapidly across the cornea over several days to 2 weeks. The epithelial rejection line can also form a ring, concentric with the limbus, that begins peripherally at the graft-host junction and progresses by shrinking centrally to a point. Another type of epithelial rejection is characterized by the presence of subepithelial infiltrates. These infiltrates contain leukocytes and frequently have an appearance similar to the subepithelial infiltrates seen in adenoviral keratoconjunctivitis.

Stromal rejection generally accompanies endothelial rejection, and is characterized by peripheral full-thickness haze with limbal injection in a previously clear graft. An arc-shaped infiltrate can be present peripherally at the graft-host junction that progresses centrally. Endothelial rejection typical results in an endothelial rejection line (Khodadoust line) that usually begins at a vascularized portion of the peripheral graft-host junction and progresses, if untreated, across the endothelial surface over several days. The rejection line includes mononuclear white cells that damage endothelial cells as the line sweeps across the endothelium. An anterior chamber reaction can also be present. In addition, a donor cornea can be clear in front of the rejection line and cloudy and edematous behind it. Endothelial rejection can also be more diffuse in character, with scattered keratic precipitates and an anterior chamber reaction indicative of endothelial rejection and damage. In this type of endothelial rejection, stromal edema typically is not localized, but rather generalized throughout the graft. Thus, indicia of corneal graft rejection include, but are not limited to, the presence of keratic precipitates, an anterior chamber reaction, circumcorneal injection, subepithelial infiltrates, epithelial rejection line, increased corneal thickness (for example, >0.62 mm more than 6 week after surgery or 10% increase in thickness within a 6 week period or between two clinic visits), increased aqueous cells; presence of cells in stroma, endothelial rejection line, and regions of corneal edema.

Exemplary specific animal models of graft rejection include a rat model such as that described in Clin Exp Immunol 107:381 (1997), a mouse model such as that described in Lau et al. Br J Ophthalmol. 82(3):294-9 (1998); a rabbit model such as that described in Hunter et al. Br J Ophthalmol. 66(5):292-302 (1982); and other models, including those resembling high-risk human corneal grafting, such as that described in Hill and Maske, Transplantation 46:26-30 (1998).

The efficacy or effective amount of a neutralizing agent specific for CXCL10 for reducing ocular inflammation, reducing spread of viral infection, and extending corneal graft survival, can be confirmed using any of a variety of well-known methods. For example, animal models predictive for ocular inflammation, viral spread, or graft survival can be used to confirm the efficacy of treatment by measuring appropriate experimental endpoints, physiological indicators, or biochemical indicators which will depend on the particular animal model selected. Several animal models appropriate for accessing the effect of an agent on inflammation are described herein above. Those skilled in the art will know which animal models can be used for determining the efficacy or effective amount of a neutralizing agent specific for CXCL10 useful in a method of the invention.

A CXCL10 neutralizing agent specific for CXCL10 administered in a method of the invention can be administered prior to onset of ocular inflammation, as well as after onset of ocular inflammation. Similarly, a CXCL10 neutralizing agent specific for CXCL10 administered in a method of the invention can be administered prior to onset of viral spread, as well as after onset of viral spread. Further, a CXCL10 neutralizing agent specific for CXCL10 administered in a method of the invention can be administered prior to corneal transplantation, concurrently with corneal transplantation, as well as after corneal transplantation. The CXCL10 neutralizing agent can be contained on or within the graft to the transplanted, or can be administered to the individual receiving corneal transplantation. When a particular neutralizing agent specific for CXCL10 is administered will depend, for example, on the chemical characteristics of the agent; the disorder or condition to be treated; and the mode of administration.

In current treatment regimes for ocular inflammation, viral infection, and corneal graft transplantation more than one compound is often administered to an individual for management of the same or different aspects of the condition. Similarly, in the methods of the invention involving reducing ocular inflammation or viral spread or extending corneal graft survival following corneal transplantation, a neutralizing agent specific for CXCL10 can advantageously be formulated with a second therapeutic compound such as an anti-inflammatory compound, anti-neovascularization compound, anti-viral compound, immunosuppressive compound or any other compound that manages the same or different aspects of the disease. Such compounds include, for example, a non-steroidal anti-inflammatory, analgesic drug (NSAID); such as Voltaren Ophtha and a steroid, such as loteprednol etabonate; and an anti-angiogenic agent. Contemplated methods of reducing ocular inflammation and viral spread or extending corneal graft survival following corneal transplantation include administering a neutralizing agent specific for CXCL10 alone, in combination with, or in sequence with, such other compounds. Alternatively, combination therapies can consist of fusion proteins, where the neutralizing agent specific for CXCL10 is linked to a heterologous protein, such as a therapeutic protein.

A neutralizing agent specific for CXCL10 useful in a method of the invention generally is administered in a pharmaceutical composition. Such a pharmaceutical composition includes a neutralizing agent and further can include, if desired, an excipient such as a pharmaceutically acceptable carrier or a diluent, which is any carrier or diluent that has substantially no long term or permanent detrimental effect when administered to a mammal. An excipient is generally mixed with active compound, or permitted to dilute or enclose the active compound. A carrier can be a solid, semi-solid, or liquid agent that acts as an excipient or vehicle for the active compound. Examples of pharmaceutically acceptable carriers and diluents include, without limitation, water, such as distilled or deionized water; saline; and other aqueous media. It is understood that the active ingredients can be soluble or can be delivered as a suspension in the desired carrier or diluent.

A pharmaceutical composition further can include, if desired, one or more agents such as emulsifying agents, wetting agents, sweetening or flavoring agents, tonicity adjusters, preservatives, buffers or anti-oxidants. Tonicity adjustors useful in a pharmaceutical composition include salts such as sodium chloride, potassium chloride, mannitol or glycerin and other pharmaceutically acceptable tonicity adjustors. Preservatives useful in the pharmaceutical compositions of the invention include, without limitation, benzalkonium chloride, chlorobutanol, thimerosal, phenylmercuric acetate, and phenylmercuric nitrate. Various buffers and means for adjusting pH can be used to prepare a pharmaceutical composition, including, but not limited to, acetate buffers, citrate buffers, phosphate buffers and borate buffers. Similarly, anti-oxidants useful in the pharmaceutical compositions of the invention are well known in the art and include, for example, sodium metabisulfite, sodium thiosulfate, acetylcysteine, butylated hydroxyanisole and butylated hydroxytoluene. It is understood that these and other substances known in the art of pharmacology can be included in a pharmaceutical composition useful in the invention.

A neutralizing agent specific for CXCL10 useful in a method of the invention is administered to an individual in an effective amount. Such an effective amount generally is the minimum dose necessary to reduce inflammation in an ocular tissue. Therefore, the term “effective amount” can be a dose sufficient to reduce inflammation, for example, by at least 30%, 40%, 50%, 60%, 70%, 80%, 90% or 100%. Such a dose generally is in the range of 0.1-1000 mg/day and can be, for example, in the range of 0.1-500 mg/day, 0.5-500 mg/day, 0.5-100 mg/day, 0.5-50 mg/day, 0.5-20 mg/day, 0.5-10 mg/day or 0.5-5 mg/day, with the actual amount to be administered determined by a physician taking into account the relevant circumstances including the severity of the ocular inflammation, the age and weight of the patient, the patient's general physical condition, the cause of ocular inflammation and the route of administration. Where repeated administration is used, the frequency of administration depends, in part, on the half-life of the neutralizing agent. It is understood that slow-release formulations also can be useful in the methods of the invention.

Various routes of administration can be useful for reducing ocular inflammation or viral spread according to a method of the invention. Routes of administration suitable for the methods of the invention include both systemic and local administration. As non-limiting examples, a pharmaceutical composition useful for reducing ocular inflammation or viral spread can be administered by topical drops, creams, gels or ointments; orally; by subcutaneous pump; by dermal patch; by intravenous or subcutaneous injection; by implanted or injected extended release formulation; and by implanted device. It is understood that the frequency and duration of dosing will be dependent, in part, on the effect desired and the half-life of the neutralizing agent.

Topical ophthalmic compositions can be useful in the methods of the invention for reducing ocular inflammation or viral spread and include, without limitation, ocular drops, ocular ointments, ocular gels and ocular creams. Such ophthalmic compositions are easy to apply and deliver the active ingredient effectively and avoid possible systemic side effects.

A preservative can be included, if desired, in an ophthalmic composition useful in the invention. Such preservatives include, without limitation, benzalkonium chloride, chlorobutanol, thimerosal, phenylmercuric acetate, and phenylmercuric nitrate. Vehicles useful in a topical ophthalmic composition include, yet are not limited to, polyvinyl alcohol, povidone, hydroxypropyl methyl cellulose, poloxamers, carboxymethyl cellulose, hydroxyethyl cellulose and purified water.

A tonicity adjustor can be included, if desired, in an ophthalmic composition used in a method of the invention. Such a tonicity adjustor can be, for example, a salt such as sodium chloride, potassium chloride, mannitol or glycerin, or another pharmaceutically or ophthalmically acceptable tonicity adjustor.

Various buffers and means for adjusting pH can be used to prepare an ophthalmic composition useful in the invention, provided that the resulting preparation is ophthalmically acceptable. Such buffers include, without limitation, acetate buffers, citrate buffers, phosphate buffers and borate buffers. It is understood that acids or bases can be used to adjust the pH of the composition as needed. Ophthalmically acceptable antioxidants useful in preparing an ophthalmic composition include, yet are not limited to, sodium metabisulfite, sodium thiosulfate, acetylcysteine, butylated hydroxyanisole and butylated hydroxytoluene.

It is understood that modifications that do not substantially affect the activity of the various embodiments of this invention are also included within the definition of the invention provided herein. Accordingly, the following examples are intended to illustrate but not limit the present invention.

### EXAMPLE I

### Neutralization of CXCL10 Prolongs Survival of HSV-1-Infected Animals

This example shows that neutralizing CXCL10 in HSV-1-infected animals prolongs survival.

To determine the effect of neutralizing CXCL10 on survival of HSV-1-infected animals, mice (n=30/group) were infected with HSV-1 (300 pfu/eye) and inoculated with 100 μg of anti-CXCL10 IgG or control IgG at time 0 and 2 and 5 days post infection. Specifically, Female ICR mice (25-30 grams, Harlan Sprague-Dawley, Indianapolis, Ind.) were anesthetized by injection with 0.1 ml of PBS containing xylazine (2 mg/ml; 6.6 mg/kg) and ketamine (30 mg/ml; 100 mg/kg) i.p. Corneas were scarified with a 25-gauge needle, and tear film was blotted with tissue before inoculating with 300 plaque forming units (pfu)/eye HSV-1. At the time of infection and 2 and 5 days post infection, mice received 100 μg anti-CXCL10 IgG or control IgG i.p. Mice were either monitored for cumulative survival over 30 days post infection (p.i.) or euthanized at the indicated time for viral titers or detection of viral antigen expression of infected tissue.

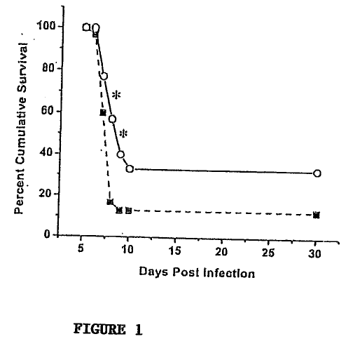

In the results shown in FIG. 1, anti-CXCL10 IgG treatment is shown as open circles and control IgG treatment is shown as black squares; and * indicates p<0.05, comparing the control to the anti-CXCL10-treated HSV-1-infected mice as determined by the Mann-Whitney U test. FIG. 1 summarizes 6 experiments performed using 5 mice per group per experiment.

As shown in FIG. 1, HSV-1-infected mice treated with anti-CXCL10 antibody exhibited prolonged survival (days 8-9 p.i.) with a trend (p=0.0547) for enhanced cumulative survival compared to the control-treated mice. This result was unexpected because previously it was determined that CXCL10 gene expression was up-regulated by in situ transfection of the cornea with the murine IFN-α1 transgene, and such treatment enhanced survival of mice infected with HSV-1 (Noisakran and Carr, J. Immunol. 164:6435 (2000)). Moreover, it was previously determined that CXCL10 exhibits anti-viral activity (Mahalingam et al., J. Virol. 73:1479 (1999)). Thus, increased survival of HSV-1-infected mice treated with anti-CXCL10 was a surprising and unexpected result in view of previously observed affects of neutralizing CXCL10.

The anti-CXCL10 mouse monoclonal antibody (clone IR7C6) was generated by immunizing BALB/c mice with a peptide corresponding to an epitope of CXCL10 (CIHIDDGPVRMRAIGK) previously shown to produce antibodies that effectively neutralize CXCL10 function in vivo (Liu et al., J. Immunol. 167:4091 (2001)). Spleens from immunized mice were removed and fused with SP2/0 myeloma cells using polyethylene glycol. Hybridoma cell lines that produced antibodies against CXCL10 were selected by ELISA and cloned twice by limiting dilution. Anti-CXCL10 hybridoma clones were selected based on their ability to recognize full length CXCL10 protein via ELISA and their viability in culture. Clone IP6C7 was chosen and produces a mAb that is an IgG2 isotope, K light chain. The hybridoma was grown in Dulbecco's modified Eagle medium (DMEM) supplemented with 10% FBS, gentamicin (Invitrogen, Carlsbad, Calif.) and antibiotic/antimycotic solution (Invitrogen, Carlsbad, Calif.) at 37° C., 5% CO2, and 95% humidity. Supernatant was collected from the hybridoma cultures and the IgG fraction was purified using protein G columns according to the manufacturer's suggestions (Pierce, Rockford, Ill.). IP6C7 effectively blocks T cell chemotaxis in vitro and inhibits Ca²⁺ mobilization.

In summary, this example shows that neutralization of CXCL10 unexpectedly prolongs survival of HSV-1-infected mice.

### EXAMPLE II

### Neutralization of CXCL10 Reduces Ocular Pathology in HSV-1-Infected Animals

This example shows that neutralizing CXCL10 in HSV-1-infected animals reduces infiltration of leukocytes into the corneal stroma, ciliary body and iris.

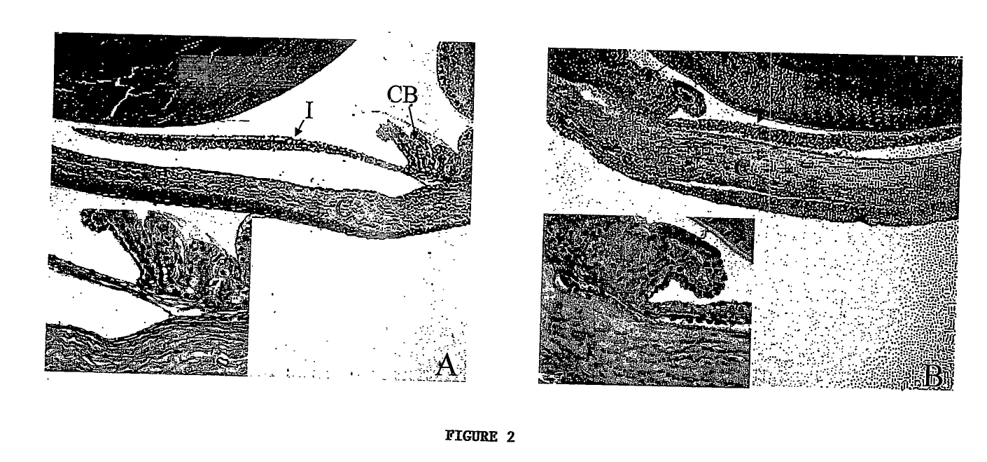

To determine the effect of neutralizing CXCL10 on ocular pathology, mice (n=8/group) were infected with HSV-1 (300 pfu/eye) and inoculated with 100 μg of anti-CXCL10 IgG or control IgG at time 0 and 2 and 5 days post infection. At day 6 p.i., the mice were euthanized and the eyes were removed and processed for hematoxylin-eosin staining. In FIG. 2A, a representative anti-CXCL10 Ab-treated mouse eye section is shown at 40× magnification; structures are labeled as follows: C is cornea; I is iris, and CB is ciliary body. In FIG. 2B, a representative control Ab-treated mouse eye section is show at 40× magnification; structures are labeled the same as in FIG. 2A. Insets for FIGS. 2A and 2B depict ciliary body and stroma at 400× magnification.

HSV-1-infected mice treated with anti-CXCL10 or control IgG also were inspected for gross pathology by physical exam using a slit lamp at a time reflecting maximum inflammation but prior to the initiation of mortality (day 5-6 p.i.). Specifically, the corneas of mice were observed for pathology by a “masked” ophthalmologist using a Kowa portable slit lamp (Kowa Optimed Inc., Torrance, Calif.). Corneal pathology assessed using a slit lamp was keyed as follows: 0=no pathology; 1=injected eye, no opacity; 2=focal opacity; 3=hazy opacity over entire cornea; 4=dense opacity in central cornea with remainder hazy; 5=same as #4 but with ulcer, and 6=corneal perforation. Control-treated mice (n=10) presented with a clinical score ranging from 2.1-2.2+/−0.3 in either the left or right eye. In contrast, the anti-CXCL10-treated mice (n=10) presented with a clinical score ranging from 0.5-0.8+/−0.3 in either the left or right eye (p<0.01, comparing the control- to anti-CXCL10-antibody treated group.) Histological assessment of the eye 6 days p.i. confirmed the majority (5/8) of anti-CXCL10 antibody-treated mice infected with HSV-1 showed modest inflammation in the cornea, proximal to the iris and ciliary body (FIG. 2A). In contrast, a majority (6/8) of control-treated, HSV-1-infected mice showed an impressive cellular infiltrate in the iris and ciliary body as well as the stroma, proximal to the ciliary body (FIG. 2B).

To further define the inflammatory process within the stroma, corneal buttons were removed from the treated mice at 3 or 6 days p.i. and CXCR3, ICAM-1, and IL-12p40 transcript expression was determined by extracting RNA from the corneal buttons, followed by real time PCR analysis. The results of these experiments are shown in Table 1. As shown, by day 6 p.i., only ICAM-1 mRNA levels were reduced in the corneal buttons from the anti-CXCL10 antibody-treated mice (Table 1).

TABLE 1
Expression of Genes Associated with Ocular Inflammation
During HSV-1 Infection

| | | Treatment | |
|---|---|---|---|
| Day PI | Transcript | anti-CXCL10 Ab | Control Ab |
| 3 | CXCR3 | 18.4 +/− 5.7^{a}* | 43.2 +/− 9.3 |
| | ICAM-1 | 2.4 +/− 0.9* | 6.3 +/− 1.1 |
| | IL-12 | 4.9 +/− 2.3 | 6.4 +/− 3.7 |
| 6 | CXCR3 | 6.5 +/− 0.5 | 10.9 +/− 2.4 |
| | ICAM-1 | 1.8 +/− 0.4* | 4.0 +/− 1.1 |
| | IL-12 | 2.2 +/− 0.4 | 2.3 +/− 0.6 |

^{a}Numbers are in relative value +/− SEM.
*p < .05 comparing the anti-CXCL10 to control antibody-treated groups.

Real time PCR analysis was performed as follows: Total RNA was extracted from corneal buttons in Ultraspect RNA isolation reagent™ (Biotecx Inc., Houston, Tex.) according to the manufacturer's protocol. First strand cDNA was synthesized using avian myoblastosis virus reverse transcriptase and an oligonucleotide dT primer (Promega, Madison, Wis.). Semiquantitative real time PCR was carried out in 96-well PCR plates (Bio-Rad, Hercules, Calif.) using a Bio-Rad iCycler. Real time PCR conditions for ICAM-1 included an initial ramp of 50° C. for 2 min followed by a denaturing step for 10 min at 95° C. followed by 40 cycles at 95° C. for 15 sec and annealing/elongation at 62° C. for 1 min. Each reaction contained 25 μl of Bio-Rad Supermix, 22.5 μl filtered water, and 2.5 μl cDNA sample. Oligonucleotide sequences for ICAM-1 include the forward primer, 5′-AGGTATCCATCCATCCCAGAGA-3′ (SEQ ID NO:1) and reverse primer, 5′-GAGCTCATCTTTCAGCCACTGA-3′ (SEQ ID NO:2). The specificity of the primer pair is described in (Moore et al., Invest. Ophthalmol. Vis. Sci. 43:2905 (2002)). The conditions and oligonucleotide sequences used to detect the housekeeping gene glyceraldehyde 3-phosphate dehydrogenase are described in (Härle et al., J. Virol. 76:6558 (2002)). Murine IL-12p40 and CXCR3 mRNA levels were measured using FAM-labeled Taqman probes and oligonucleotide sequences according to the manufacturer's instructions (Roche, Branchburg, N.J.). The PCR results were analyzed on the iCycler software (version 3.0) and threshold-cycles were determined as described in (Härle et al., supra, (2002)).

Selective chemokine protein levels also were assessed during the initial stage of infection. Mice (n=5/group) were infected with HSV-1 (300 pfu/eye) and inoculated with 100 μg of anti-CXCL10 IgG or control IgG at time 0 and 2 and 5 days post infection. At day 5 or 7 p.i., the mice were euthanized and the corneal buttons and iris from each eye was removed and homogenized. The clarified supernatant from each sample (n=10/group day 5 p.i.; n=4/group day 7 p.i.) was then assayed for chemokine content by ELISA.

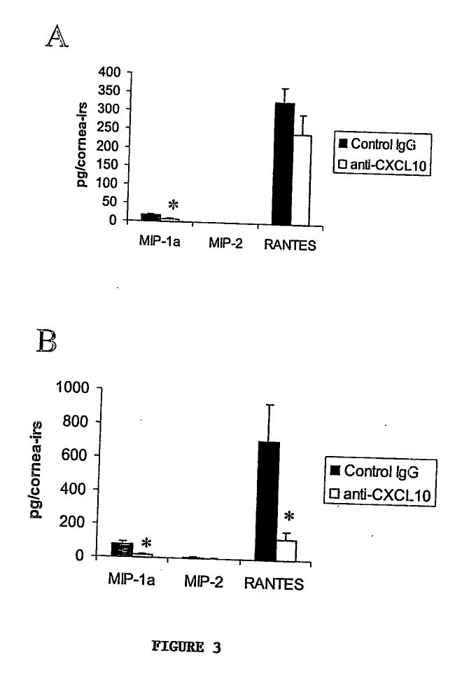

As shown in FIG. 3A, on day 5 p.i., MIP-1 levels in the cornea and iris were reduced 3-fold in the anti-CXCL10-treated group in comparison to the control-treated mice. The prominent expression of RANTES while reduced in the anti-CXCL10-treated mice, was not significantly different in comparison to the control-treated mice at day 5 p.i. (FIG. 3A). MIP-2 levels were below the level of detection (<15.6 pg) in most animals (7/10) of either group of treated mice at this time point. By day 7 p.i., both RANTES and MIP-1α but not MIP-2 levels were significantly reduced in the cornea/iris of the anti-CXCL10-treated HSV-1-infected mice in comparison to the control-treated group (FIG. 3B). There was no measurable MIP-1α, MIP-2, or RANTES in non-infected ocular tissue. Anti-CXCL10 antibody treatment reduced MIP-1α and RANTES levels in the cornea/iris of HSV-1 infected mice. In FIG. 3, * indicates p<0.05 comparing the anti-CXCL10 treated group to the corresponding control-treated group as determined by ANOVA and Tukey's post hoc t-test.

For this and the following examples, the significance of differences (p<0.05) between the viral titers, clinical scores, and relative values for targeted gene expression recovered from the corneal buttons, iris, retina, and TG of control (IgG)- and anti-CXCL10-treated mice were determined by one-way ANOVA and Tukey's test. The Mann-Whitney U test was used to determine the significant (p<0.05) difference in the cumulative survival studies. All statistical analysis was performed using the GBSTAT program (Dynamic Microsystems, Silver Spring, Md.).

The reduced inflammatory profile in the cornea of HSV-1-infected mice treated with anti-CXCL10 Ab was most closely associated with a reduction in the chemokine MIP-1α and the expression of ICAM-1 mRNA. These results are consistent with previously reported observations (Tumpey et al., J. Virol. 72:3705 (1998) and Tumpey et al., J. Leukoc. Biol. 63:4486 (1998)) highlighting the key importance of MIP-1α in the inflammatory process associated with ocular HSV-1 infection. Another chemokine, RANTES, was reduced in the anti-CXCL10 Ab-treated mice specifically at the latter sampling point (day 7 p.i.) in both the cornea/iris and TG. The amount of RANTES recovered in the cornea and iris of HSV-1-infected mice was nearly 10-fold greater than that of MIP-1α implicating this chemokine in the pathological manifestations of ocular herpes infection that typically occurs after the initial clearing of virus from the eye; 14-21 days p.i. (Tumpey et al., supra (1998)). RANTES is chemoattractant for monocytes as well as T cells (Rollins, B. J., Blood 90:909 (1997)) implicating this CC chemokine as another instigator along with CD4+ T cells in herpes keratitis. Although CXCL10, MIP-2, and RANTES mRNA are constitutively expressed in the cornea of mice (Su et al., supra (1996)), the levels must be below that which we can detect by ELISA. Similar to the effects on MIP-1α and RANTES, treatment of HSV-1-infected mice with anti-CXCL10 Ab also reduced ICAM-1 mRNA expression which is another molecule associated with inflammation following ocular HSV-1 infection (Dennis et al., Current Eye Res. 14:55 (1995)). Consequently, the additive effect of suppressing the expression of both adhesion molecules as well as chemokines could ultimately reduce the marked inflammatory process normally observed during acute HSV-1 disease.

To confirm that treatment of mice with CXCL10 neutralizing antibody reduced tissue levels of CXCL10, CXCL10 protein was measured at times post HSV-1 infection. Female ICR mice (n=6/group) were infected with HSV-1 (300 pfu/eye) and inoculated with 100 μg of anti-CXCL10 IgGl or control IgG at time 0 and 2 and 5 days post infection. At day 1, 3, or 6 post infection, the mice were euthanized and the eyes and trigeminal ganglia (TG) were removed and assayed for chemokine content by ELISA.

As shown in Table 2, during the early period of acute infection (i.e., day 1-3 p.i.), mice receiving anti-CXCL10 showed a significant reduction in CXCL10 expression in the eye compared to control IgG-treated, HSV-1 infected mice. The chemokine MIG, which is induced by IFN-γ and involved in Th1-directed inflammatory responses, was not significantly reduced in the eye of anti-CXCL10 Ab-treated mice. Another difference in ocular levels of CXCL10 and MIG is that there are detectable levels of CXCL10 in uninfected mice whereas MIG levels are only detected in the eye of mice infected with HSV-1. Whereas CXCL10 and MIG levels were not significantly different in the TG during the early course of infection (days 1-3), by day 6 p.i., anti-CXCL10 Ab-treated mice showed a significant reduction of both chemokines in the TG compared to control IgG-treated animals.

TABLE 2
Treatment of Mice with Anti-CXCL10 Antibody
Suppresses Tissue Levels of CXCL10 During HSV-1 Infection

| | | | Treatment | |
|---|---|---|---|---|
| Day PI | Tissue | Chemokine | anti-CXCL10 Ab | Control Ab |
| 1 | Eye | CXCL10 | 0.0 +/− 0.0*^{a} | 72 +/− 26 |
| 3 | Eye | CXCL10 | 28 +/− 19* | 110 +/− 32 |
| 6 | Eye | CXCL10 | 532 +/− 224 | 934 +/− 321 |
| 1 | Eye | MIG | 0.0 +/− 0.0 | 0.0 +/− 0.0 |
| 3 | Eye | MIG | 34 +/− 14 | 82 +/− 23 |
| 6 | Eye | MIG | 1331 +/− 323 | 1128 +/− 342 |
| 1 | Eye | VEGF | 7.4 +/− 3.3 | 8.0 +/− 1.6 |
| 3 | Eye | VEGF | 5.7 +/− 1.2* | 10.1 +/− 1.6 |
| 6 | Eye | VEGF | 4.2 +/− 2.0 | 5.6 +/− 2.2 |
| 1 | TG | CXCL10 | 0.0 +/− 0.0 | 0.0 +/− 0.0 |
| 3 | TG | CXCL10 | 111 +/− 55 | 287 +/− 158 |
| 6 | TG | CXCL10 | 807 +/− 225* | 1466 +/− 131 |
| 1 | TG | MIG | 0.0 +/− 0.0 | 0.0 +/− 0.0 |
| 3 | TG | MIG | 128 +/− 67 | 77 +/− 29 |
| 6 | TG | MIG | 681 +/− 164* | 1520 +/− 180 |

^{a}Numbers are in pg/tissue +/− SEM.
*p < .05 comparing the anti-CXCL10 to control antibody-treated groups. This table is a summary of two experiments with n = 3 mice/group/experiment.

In summary, this example shows that anti-CXCL10 antibody treatment reduces infiltration of leukocytes into the corneal stroma, ciliary body, and iris of HSV-1 infected mice, and reduces MIP-1α and RANTES levels in the cornea/iris of HSV-1 infected mice.

### EXAMPLE III

### Neutralization of CXCL10 Enhances HSV-1 Replication but Hinders HSV-1 Trafficking to the Retina

This example shows that neutralizing CXCL10 in HSV-1-infected animals hinders HSV-1 spread to the retina.

As described herein above, treatment of HSV-1-infected mice with anti-CXCL10 antibody caused a reduction in leukocyte infiltration in the stroma, ciliary body, and iris following corneal HSV-1 infection. To determine viral titers in ocular tissues of anti-CXCL10 and control IgG-treated mice, samples were taken during the acute viral infection.

To determine viral titers, plaque assays were performed. At day 3, 5, or 7 days p.i., virally-infected mice were euthanized and the corneal buttons, iris, retina, and trigeminal ganglion (TG) were isolated and homogenized in 1.0 ml of RPMI-1640. The clarified supernatant (1000×g, 1 min) from the homogenized tissue was serially diluted and placed (100 μl) onto Vero cell monolayers in 96-well cultured plates. After a 1 hr incubation at 37° C., 5% CO₂, 95% humidity, the supernatants were discarded, and 75 μl of an overlay solution (0.5% methylcellulose in culture medium) was added on top of the monolayers. The cultures were incubated at 37° C. in 5% CO2 and 95% humidity for 24-28 hours to observe plaque formation, and the amount of infectious virus was reported as pfu/ml. In the experiments described below in Example IV, CD11b⁺ or CD11b⁻-enriched cells (60-2500 cells/well) were added to Vero cell monolayers and observed for plaque formation 28 hours post initiation of culture.

As shown in Table 3, at day 3 p.i., viral titers were elevated in the corneal buttons taken from anti-CXCL10 Ab-treated mice. However, by day 5 p.i., there was no significant difference in viral titers recovered from the cornea By comparison, HSV-1 was initially recovered in the iris of control—but not anti-CXCL10 Ab-treated mice as early as day 3 p.i. By day 5 p.i., there was a significant increase in the amount of HSV-1 recovered in the iris of the control-treated mice compared to the anti-CXCL10 Ab-treated group. By day 7 p.i., both groups of animals had significant amounts of infectious virus in the iris clearly above that recovered in the corneal buttons. Retinal tissue was employed as a negative control since earlier work suggested that the ipsilateral retina is spared from HSV-1 following anterior segment infection (Azumi and Atherton, Invest. Ophthalmol. Vis. Sci. 35:3251 (1994)).

Although no virus was recovered early during infection (i.e., 0/8 per group, day 3 p.i.), by day 5 p.i., 12/20 retinas surveyed from control Ab-treated mice were positive for infectious virus compared to 1/12 retinas from the anti-CXCL10 Ab-treated group (Table 3). By day 7 p.i., there was a significant increase in viral titers recovered from retina of the control-treated group in comparison to the anti-CXCL10-treated mice (Table 3).

TABLE 3
Anti-CXCL10 Treatment Hinders HSV-1 Spread to the Retina

| | | Treatment | Control |
|---|---|---|---|
| Day PI | Tissue | anti-CXCL10 Ab | Antibody |
| 3 | Cornea | 4,054 +/− 932^{a}* | 1,359 +/− 321 |
| 5 | Cornea | 17,567 +/− 11,611 | 99,430 +/− 41,585 |
| 7 | Cornea | 736 +/− 289 | 1,974 +/− 898 |
| 3 | Iris | 0 +/− 0 | 50 +/− 25 |
| 5 | Iris | 17,254 +/− 7,475* | 144,607 +/− 56,698 |
| 7 | Iris | 91,938 +/− 56,693 | 672,055 +/− 408,184 |
| 3 | Retina | 0 +/− 0 | 0 +/− 0 |
| 5 | Retina | 10 +/− 10 | 61 +/− 27 |
| 7 | Retina | 119 +/− 91* | 5,318 +/− 2,000 |
| 3 | TG | 3,716 +/− 2322 | 5,421 +/− 1,521 |
| 5 | TG | 21,696 +/− 4,008* | 9,030 +/− 2,456 |

^{a}Numbers indicate viral titer in pfu/corneal button, iris, retina, or trigeminal ganglion (TG) +/− SEM.
*p < .05 comparing the control- to the anti-CXCL10 Ab-treated groups for each tissue at each time point.

It is evident that viral titers recovered in the eye and TG do not reflect the outcome of survival of HSV-1-infected mice (see Example I), since the anti-CXCL10 Ab-treated mice showed prolonged survival even though viral yields were significantly greater in the infected tissue surveyed.

Since viral yields in the eye and TG of anti-CXCL10 Ab-treated mice were greater than those found in the control Ab-treated animals at day 3 and day 5 p.i. respectively, the direct effect of CXCL10 on HSV-1 replication was tested using primary murine spleen cells or the fibroblast cell line, L929. Spleen cells (1×10⁷ cells/culture) of L929 (1.6×10⁶ cells/culture) cells were pre-treated with CXCL10 (0.5-1.0 μg/culture) or vehicle (RPMI-1640) for 24 hr prior to the addition of HSV-1 (MOI=0.5 for spleen cells and 1.0 for L929 cells). Twenty four hours post infection, the cultures were freeze-thawed and the cell-free supernatants were assayed for viral titer using Vero cells. The results shown in Table 4 represent two experiments, n=2 cultures/treatment/experiment. As the data in Table 4 indicate, preincubating spleen or L929 cells with CXCL10 prior to infection with HSV-1 (McKrae strain) had no direct effect on viral replication. Therefore, CXCL10 does not effect viral replication as measured by viral titer.

TABLE 4
CXCL10 Does Not Directly Effect HSV-1
Replication

| Cells | CXCL10 | Vehicle |
|---|---|---|
| L929 | 3,471,875 +/− 234,375^{a} | 2,968,750 +/− 156,250 |
| Spleen | 17,188 +/− 1,563 | 18,750 +/− 3,125 |

^{a}Numbers are in mean PFU/ml +/− SD

In summary, this example shows that neutralizing CXCL10 enhances HSV-1 replication but hinders HSV-1 trafficking to the retina.

### EXAMPLE IV

### Immunohistochemical Staining of HSV-1 Antigen in the Eve

This example shows that neutralizing CXCL10 in HSV-1-infected animals results in reduced HSV-1 antigen expression in ocular tissues.

Ocular tissue was assessed for viral antigen expression and location comparing the control IgG-treated mice to that of the anti-CXCL10-treated group. Mice (n=4/group) were infected with HSV-1 (300 pfu/eye) and inoculated with 100 μg of anti-CXCL10 IgG or control IgG at time 0 and 2 and 5 days post infection. At day 6 p.i., the mice were euthanized and the eyes were removed and processed for HSV-1 antigen expression. The experiment was repeated twice.

Ocular tissue expression of HSV-1 antigen and detection of cellular infiltration in the cornea and retina of infected eyes was carried out as described in (Noisakran and Carr supra (2000)). Whole mount staining for HSV-1 Ag expression was performed as follows. Whole eyes were fixed in 4% paraformaldehyde overnight. The retinas were then dissected away from the sclera and choroid, and placed in separate tubes. Tissues were then washed in PBS-T (PBS containing 1% Triton X-100), then blocked in PBS-T containing 10% horse serum for 30 minutes at room temperature (25° C.). Tissues were then incubated with an FITC conjugated anti-HSV1 antibody (DAKO, F0318, Carpinteria, Calif.), diluted 1:200 in PBS-T containing 10% horse serum for four hours at room temperature. Tissues were then washed with PBS-T three times for 15 minutes each wash, then flat mounted in 50% glycerol in PBS onto microscope slides. Tissues were then imaged on a Nikon fluorescent microscope equipped with a high-resolution digital camera.

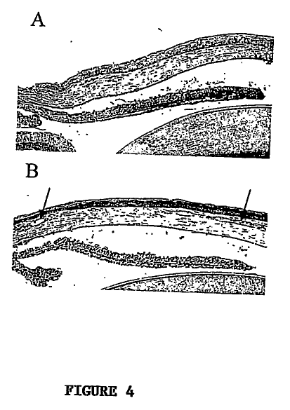

FIG. 4A shows IgG-treated mouse iris at a magnification of 100×. FIG. 4B shows anti-CXCL10 antibody-treated mouse iris at a magnification of 100×. Note the dark-stained tissue indicative of HSV-1 antigen and the edematous presentation of the corneal stroma of the control IgG-treated mice relative to the anti-CXCL10 IgG-treated animals.

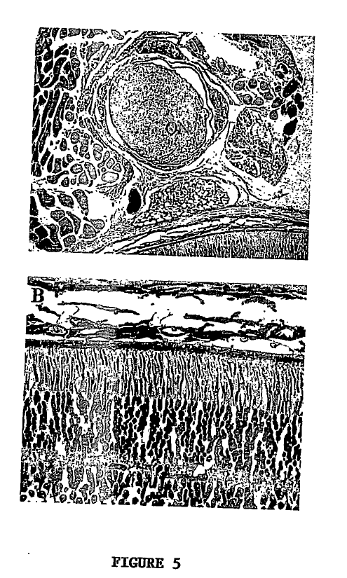

FIG. 5 is a color photograph that shows viral antigen expression in the retina of mice infected with HSV-1. Mice (n=4/group) were infected with HSV-1, euthanized 6 days p.i., and the eyes removed and processed for HSV-1 antigen expression. FIG. 5A shows HSV-1 antigen expression in tissue surrounding the optic nerve (ON). FIG. 5B shows HSV-1 antigen expression (indicated by arrows) in the choroid and photoreceptor layer of the retina.

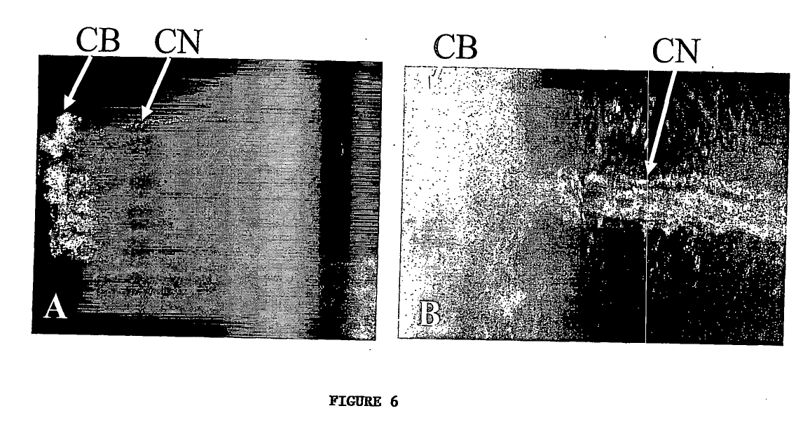

FIG. 6 shows expression of HSV-1 antigen in the ciliary body and nerve of mice 6 days p.i. Whole mounts of ocular tissue were incubated with rabbit anti-HSV-1 polyclonal antibody. After excessive washing, the tissue was viewed under a fluorescence microscope. FIG. 6A shows the ciliary body (CB) and nerve (CN) labeled with HSV-1 Ag (Magnification is 40×). FIG. 6B shows the same image as FIG. 6B, but at 200× magnification.

As indicated by FIG. 4, both groups of mice expressed HSV-1 antigen in the cornea and iris with more viral antigen expression in the iris of control-treated mice. Likewise, as shown in FIG. 5, viral antigen was expressed in select areas adjacent to or around arterioles and within the choroid and in the retina typically the ganglion cell layer and in the inner plexiform layer of the retina. In addition, as shown in FIG. 6, viral antigen expression was detected in the ciliary nerve of HSV-1 infected mice 6 days p.i.

Since anti-CXCL10 antibody-treated mice presented with a reduced cellular infiltrate within the corneal stroma proximal to the iris and ciliary body along with a delay in infectious virus recovered in the retina compare to control-treated, HSV-1-infected mice, it was anticipated that inflammatory cells might contribute to the infection of the retina. In fact, occasional cellular infiltrate in the vitreous of HSV-1-infected mice expressed HSV-1 antigen.

To determine if such cells harbored infectious virus, cells isolated from the vitreous humor were enriched for the CD11b⁺ phenotype and assayed for viral content by plague assay. For these experiments, five days following ocular infection with HSV-1 (300 pfu/eye), the eyes were removed and the vitreous was collected. The contents of the vitreous were washed in PBS (pH 7.4) and the resulting cells were placed in 500 μl of degassed PBS containing 2 mM EDTA and 0.5% BSA (Sigma Chemical Co., St. Louis, Mo.). The cells were then positively selected for CD11b³⁰ cells by using LS⁺ separating columns and CD11b magnetic microbeads according to the manufacturer's instructions (Miltenyi Biotech, Auburn, Calif.). Following the separation of CD11b⁺ and CD11b⁻ cells, the recovered cells were counted and assayed for HSV-1 content by plaque assay. In order to obtain enough cells to conduct a plaque assay, the vitreous from 20 eyes/experiment was employed recovering approximately 30,000 total mononuclear cells.

More than 50 pfu were obtained from 62 CD11b⁺ cells compared to 15+/−12 pfu from 62 CD11b⁻ cells suggesting that the CD11b⁺ cells harbor the majority of infectious HSV-1 in the vitreous. There was no significant difference in the number of cells recovered in the vitreous of HSV-1 infected mice treated with control antibody (4.3+/−0.9×10⁴ cells) compared to mice treated with the anti-CXCL10 antibody (3.3+/−1.6×10⁴ cells) suggesting the virus may infect the retina by additional routes independent of inflammatory cells (for example, the ciliary nerve).

In summary, this example shows that HSV-1 antigen expression in the iris is reduced in anti-CSCL10 antibody-treated HSV-1-infected mice.

### EXAMPLE V

### Corneal Scarification is Required for HSV-1 Presence in the Retina

This example shows that HSV-1 infection of the retina is not strain-dependent.

Since HSV-1 in the retina had not been previously reported following corneal infection, an experiment was undertaken to determine the requirement for HSV-1 access to the retina. Mice were infected with the McKrae strain of HSV-1 either with (200 pfu/eye) or without (70,000 pfu/eye) scarification and assessed for viral titers in the corneal buttons, iris, and retina at day 5 p.i.

As indicated by the results shown in Table 5, virus was recovered in the retina only when the cornea was scarified, even when over 100-fold more virus was applied to the non-scarified cornea compared to the scarified cornea. In addition, there was a significant reduction in the viral load in the iris from mice in which virus was applied to the non-scarified cornea suggesting scarification augments the access to internal sites within the anterior segment of the eye.

In these experiments, mice (n=4/group) were infected with McKrae (200 pfu/eye, scarified; 70,000 pfu/eye, non-scarified) or KOS (70,000 pfu/eye scarified or non-scarified) strains of HSV-1. Five days p.i., the mice were euthanized and the designated tissue was removed using a dissecting microscope, homogenized, and assayed for infectious virus by plaque assay.

TABLE 5
Corneal Scarification is Required for HSV-1 Infection of Retina

| | McKrae | | KOS | |
|---|---|---|---|---|
| Tissue | Scarified | Non-scarified | Scarified | Non-scarified |
| Cornea | 4,430 +/− 1,190^{a} | 70,830 +/− 37,030 | 8,000 +/− 4,910 | 1,700 +/− 870 |
| Iris | 53,750 +/− 17,330 | 450 +/− 450* | 117,500 +/− 40,430 | 50 +/− 30* |
| Retina | 75 +/− 48 | 0 +/− 0 | 830 +/− 700 | 0 +/− 0 |

^{a}Numbers indicate viral titer in pfu/corneal button, iris, or retina +/− SEM.
*p < .05 comparing the scarified- to non-scarified groups for each strain in the iris.

As described above, it was found that HSV-1 traffics to the retina upon topical application onto the scarified cornea. Previous studies have demonstrated the retrograde transport of HSV-1 following introduction of the virus onto scarified corneal epithelium but targeted tissue has focused on cells within the peripheral and central nervous systems excluding retinal tissue (Shimeld et al., J. Virol. 75:5252 (2001)). Other investigators have shown the zosteriform spread of HSV-1 during acute ocular infection involves the TG requiring replication in the nervous system rather than corneal tissue (Summers et al., J. Virol. 75:5069 (2001)). Clinically, HSV-1 has been reported to infect the contralateral retina via the brain following anterior chamber inoculation (Atherton and Streilein, Invest. Ophthalmol. Vis. Sci. 28:571 (1987)). In this experimental model, the ipsilateral retina is protected by T cells and NK cells (Igietseme et al., J. Virol. 65:763 (1991); Azumi and Atherton, Invest. Ophthalmol. Vis. Sci. 35:3251 (1994) and Tanigawa et al., Invest. Ophthalmol. Vis. Sci. 41:132 (2000)). It has also been reported that the presence of an HSV-1 ribonucleotide reductase mutant in the retina following the application of the virus onto the scarified cornea of a mouse (Spencer et al., Invest. Ophthalmol. Vis. Sci. 25 41:1392 (2000)).

Although there was no indication by what mechanism the virus applied to the scarified cornea spread to the retina, the present study found CD11b⁺ and CD11b− cells within the vitreous to harbor infectious virus. In addition to inflammatory cells, another conduit by which the virus reaches the retina could be the ciliary nerve, which was found to stain positive for HSV-1 antigen in mice infected 6 days earlier. In humans, the ciliary ganglia has also found to possess HSV-1 DNA implicating this tissue in addition to the more traditional TG as a site of HSV-1 latency (Bustos and Atherton, Invest. Ophthalmol. Vis. Sci. 43:2244 (2002)).

In summary, this example shows that HSV-1 infection of the retina was not strain-dependent, as both the McKrae strain and KOS strain of HSV-1 could readily infect retinal tissue when applied topically to scarified but not non-scarified cornea.

### EXAMPLE VI

### Inflammation in HSV-1-Infected Trigeminal Ganglion is Reduced in Mice Treated with Anti-CXCL10 Antibody

This example shows that neutralizing CXCL10 in HSV-1-infected animals results in reduced levels of cytokines in the trigeminal ganglia (TG).

Following ocular HSV-1 infection, HSV-1 virus traffics back into the sensory ganglion where it establishes a latent infection. To determine whether mice treated with neutralizing antibody to CXCL10 showed a change in HSV-1 infection or inflammation in response to infection within the TG, the sensory ganglia of HSV-1-infected mice treated with control IgG or anti-CXCL10 IgG were removed during acute infection and assayed for viral titer and chemokine and IFN-γ protein levels.

In vivo levels of IFN-γ, MIP-1α, MIP-2, and RANTES were measured in the TG of mice infected with HSV-1 at days 5-7 p.i. using commercially available ELISA kits (R&D Systems, Minneapolis, Minn.). Mice (n=5/group) were infected with HSV-1 (300 pfu/eye) and inoculated with 100 g of anti-CXCL10 IgG or control IgG at time 0 and 2 and 5 days post infection. At day 5 or 7 p.i., the mice were euthanized and the TG were removed and placed in PBS (pH 7.4) supplemented with a cocktail of protease inhibitors (Calbiochem, San Diego, Calif.). Following homogenization, the samples (n=10/group day 5 p.i.; n=4/group day 7 p.i.) were clarified by centrifugation (10,000×g, 2 min) and the clarified supernatant was assayed for chemokine and IFN-γ content in duplicate by ELISA according to the manufacturer's instructions. Non-infected eyes served as the negative control.

As indicated by the results shown in Table 1, at day 3 p.i., there was no significant difference in viral loads comparing the control and anti-CXCL10 antibody-treated groups. In contrast, at day 5 p.i., the anti-CXCL10 antibody-treated, group showed a significant increase in viral load compared to the control-treated group.

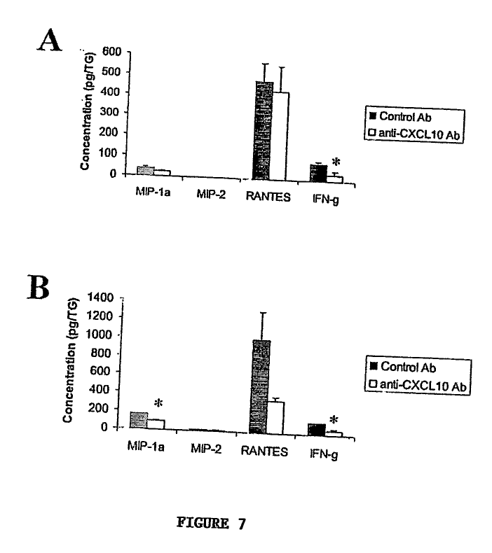

As indicated by the results shown in FIG. 7A, even though the viral yield was increased in the anti-CXCL10 antibody-treated group at day 5 p.i., IFN-γ levels in the TG at this time point were reduced. Chemokine levels were not significantly modified in the TG day 5 p.i. As indicated by the results shown in FIG. 7B, at day 7 p.i., MIP-1α and the pro-inflammatory cytokine, IFN-γ were reduced in the TG of mice treated with the anti-CXCL10 antibody.

In summary, this example indicates that, similar to ocular tissue, HSV-1 infected TG taken from mice treated with the neutralizing antibody to CXCL10 show a reduced level of inflammation as measured by MIP-1α and IFN-γ protein concentration.

### EXAMPLE VII

### Neutralization of CXCL10 Reduces Neovascularization in HSV-1-Infected Animals

This example shows that neutralizing CXCL10 in HSV-1-infected animals reduces neovascularization in the eye.

To determine the effect of anti-CXCL10 treatment on neovascularization, VEGF levels were measured in HSV-1 infected mice treated with anti-CXCL10 or control. As shown in Table 2, a transient but significant reduction in the VEGF level in the eye of anti-CXCL10 Ab-treated mice compared to the control IgG-treated group 3 days p.i. was observed. To confirm the relationship between vascular development in the eye of HSV-1 infected mice treated with anti-CXCL10 versus control IgG, neovascularization was observed in transgenic mice expressing the LacZ gene under the vascular endothelial-specific promoter, Tie2.

For these experiments, Tie2 transgenic mice were infected with HSV-1 (300 pfu/eye) and treated with 100 μg of control (n=9) or anti-CXCL10 IgG (n=10) at 0, 2, and 5 days p.i. Mice were euthanized at day 6 p.i. and the eyes were removed and enucleated. To isolate anterior segments, enucleated eyes were dissected to remove posterior segment and lens in ice-cold PBS and fixed in ice-cold 2% paraformaldehyde, 2 mM MgCl₂, 2 mM EGTA, 0.1 M Pipes buffer, pH 6.9 for 45 minutes. The fixed anterior segments were rinsed with PBS three times for 5 minutes each. The LacZ expression was detected by room temperature overnight incubation in 0.1% X-gal, 5 mM potassium ferricyanide, 5 mM potassium ferrocyanide, 1 mM magnesium chloride, 0.002% NP-40, 0.01% sodium deoxycholate, PBS, pH 7.0. After staining, the anterior segments were rinsed in PBS and post-fixed overnight at 4° C. in 4% paraformaldehyde, PBS, pH 7.0. The corneal button was then removed and flat mounts of the cornea were prepared for visualization of vascularizaiton. For whole-mount photography, the post-fixed eyes were rinsed in PBS and equilibrated in 50% glycerol, PBS. Images were captured on an Olympus SZX12 stereo microscope.

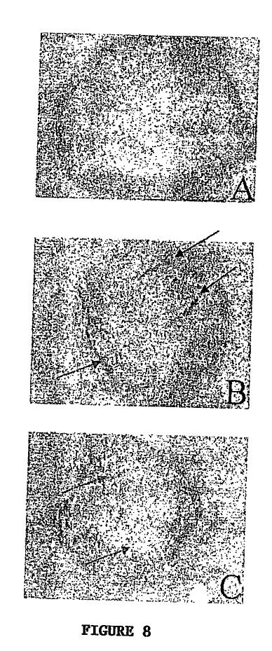

As shown in FIG. 8, vascular beds in the iris can be visualized peripheral to the cornea. Arrows indicate sites of neovascularization of the cornea. Panel A represents anti-CXCL10 IgG treated HSV-1 infected mouse showing no neovascularization. Panel B represents a control IgG-treated HSV-1 infected mouse showing neovascularization in the cornea. Panel C represents an anti-CXCL10 IgG-treated HSV-1 infected mouse also showing neovascularization in the cornea.

The majority (6/9) of HSV-1 infected, Tie2-LacZ transgenic mice treated with control Ab showed neovascularization 6 days p.i. compared to 40% (4/10) of the anti-CXCL10-treated mice. Collectively, HSV-1 infected mice treated with the anti-CXCL10 Ab show a modest reduction in the incidence or level of neovascularization.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention.

What is claimed is:

1. A method of reducing ocular inflammation in an individual susceptible to ocular inflammation, comprising administering to said individual an effective amount of a neutralizing agent specific for CXCL10.

2. The method of claim 1, wherein said individual is a mammal.

3. The method of claim 1, wherein said individual is human.

4. The method of claim 1, wherein said individual has a microbial infection.

5. The method of claim 4, wherein said microbial infection is selected from a viral infection, a bacterial infection, a fungal infection and a parasitic infection.

6. The method of claim 5, wherein said infection is a herpes virus infection.

7. The method of claim 1, wherein said ocular inflammation is corneal inflammation.

8. The method of claim 1, wherein said neutralizing agent is administered prior to onset of ocular inflammation.

9. The method of claim 1, wherein said neutralizing agent is administered after onset of ocular inflammation.

10. The method of claim 1, wherein said neutralizing agent specific for CXCL10 comprises a CXCL10 binding agent.

11. The method of claim 1, wherein said CXCL10 binding agent is an anti-CXCL10 antibody, or fragment thereof.

12. The method of claim 11, wherein said anti-CXCL10 antibody, or fragment thereof is monoclonal.

13. The method of claim 1, wherein said neutralizing agent is administered interocularly.

14. A method for reducing spread of viral infection within ocular tissues of an individual susceptible to ocular viral infection, comprising administering to said individual an effective amount of a neutralizing agent specific for CXCL10.

15. The method of claim 14, wherein said individual is a mammal.

16. The method of claim 15, wherein said individual is human.

17. The method of claim 16, wherein said viral infection is a herpes virus infection.

18. The method of claim 14, wherein said individual has a viral infection of the cornea.

19. The method of claim 18, wherein said administering reduces spread of viral infection from the cornea to the retina.

20. The method of claim 18, wherein said administering reduces spread of viral infection from the cornea to the iris.

21. The method of claim 14, wherein said neutralizing agent is administered prior to onset of spread of viral infection.

22. The method of claim 14, wherein said neutralizing agent is administered after onset of spread of viral infection.

23. The method of claim 14, wherein said neutralizing agent specific for CXCL10 comprises a CXCL10 binding agent.

24. The method of claim 23, wherein said CXCL10 binding agent is an anti-CXCL10 antibody, or fragment thereof.

25. The method of claim 24, wherein said anti-CXCL10 antibody, or fragment thereof is monoclonal.

26. The method of claim 14, wherein said neutralizing agent is administered interocularly.

27. A method of extending corneal graft survival following corneal transplantation in an individual, comprising administering to said individual an effective amount of a neutralizing agent specific for CXCL10.

28. The method of claim 27, wherein said neutralizing agent is administered prior to corneal transplantation.

29. The method of claim 27, wherein said neutralizing agent is administered after corneal transplantation.

30. The method of claim 27, wherein said neutralizing agent specific for CXCL10 comprises a CXCL10 binding agent.

31. The method of claim 30, wherein said CXCL10 binding agent is an anti-CXCL10 antibody, or fragment thereof.

32. The method of claim 31, wherein said anti-CXCL10 antibody, or fragment thereof is monoclonal.

33. The method of claim 27, wherein said neutralizing agent is administered interocularly.

34. The method of claim 27, said neutralizing agent is administered by release from an intraocular or periocular implant.

35. A method for screening for a compound for reducing ocular inflammation in an animal, comprising:
(a) providing a compound that is a neutralizing agent specific for CXCL10; and
(b) determining the ability of said compound to reduce one or more indicia of ocular inflammation,
wherein a compound that reduces one or more indicia of ocular inflammation is identified as a compound for reducing ocular inflammation in an animal.

36. The method of claim 35, wherein said compound is administered to an animal capable of exhibiting an index of ocular inflammation.

37. The method of claim 35, wherein said animal is a mammal.

38. The method of claim 37, wherein said animal is a mouse.

39. The method of claim 35, wherein said compound is contacted with a tissue capable of exhibiting an index of ocular inflammation.

40. The method of claim 35, wherein said tissue is a synthetic tissue.

41. The method of claim 35, wherein said tissue is an animal tissue.

42. The method of claim 35, wherein said neutralizing agent specific for CXCL10 comprises a CXCL10 binding agent.

43. The method of claim 42, wherein said CXCL10 binding agent is an anti-CXCL10 antibody, or fragment thereof.

44. The method of claim 43, wherein said anti-CXCL10 antibody, or fragment thereof is monoclonal.

45. The method of claim 35, wherein said one or more indicia of ocular inflammation includes an index selected from reduced corneal pathology, reduced leukocyte infiltration, reduced MIP-1α expression, reduced ICAM-1 expression, reduced CXCR3 expression, reduced RANTES expression, reduced viral antigen expression, reduced viral spread, increased survival and reduced neovascularization.


SEQUENCE LISTING (2 sequences)

SEQ ID NO: 1 - DNA, 22 bp, Artificial Sequence
  primer
aggtatccatccatcccagaga

SEQ ID NO: 2 - DNA, 22 bp, Artificial Sequence
  primer
gagctcatctttcagccactga